(19) United States
(12) Patent Application Publication
(10) Pub. No.: US 2021/0322684 A1
(43) Pub. Date: Oct. 21, 2021

(54) HEATED SURGICAL CANNULA FOR PROVIDING GASES TO A PATIENT

(71) Applicant: Fisher & Paykel Healthcare Limited, Auckland (NZ)

(72) Inventors: Christian Francis Fischer, Auckland (NZ); Richard John Boyes, Auckland (NZ); Abigail Sharmini Rajen Arulandu, Auckland (NZ); Benjamin Elliot Hardinge Pegman, Auckland (NZ); Zane Paul Gell, Auckland (NZ); Zach Jonathan Warner, Auckland (NZ); Katie-Ann Jane Buckels, Auckland (NZ); Vincent Verdoold, Auckland (NZ); Charlotte Grace Laus, Auckland (NZ)

(21) Appl. No.: 17/268,747

(22) PCT Filed: Aug. 16, 2019
(86) PCT No.: PCT/NZ2019/050101

§ 371 Date: Feb. 16, 2021

Related U.S. Application Data
(60) Provisional application No. 62/719,557, filed on Aug. 17, 2018.

(51) Int. Cl. A61M 13/00

(52) U.S. Cl. CPC A61M 13/003; A61M 2205/7536; A61M 2205/3653

(57) ABSTRACT

A surgical cannula for providing insufflation gases to a surgical cavity of a patient (for example, the pneumoperitoneum) and allowing insertion of medical instruments into the surgical cavity through the cannula can include a heater within or coupled to the cannula. The heater can heat the gases and/or the instruments to raise the temperature of the gases and/or instruments above a dew point of the gases to prevent fogging. The heater can also help to defog a lens of a medical instrument by heating to clear the lens and improve optical clarity.

### FIELD OF THE DISCLOSURE

The present disclosure relates to humidifier systems and components of humidifier systems for gases to be supplied to a patient, in particular to a patient during a medical procedure.

### BACKGROUND

Various medical procedures require the provision of gases, typically carbon dioxide, to a patient during the medical procedure. For example, two general categories of medical procedures often require providing gases to a patient. These include closed type medical procedures and open type medical procedures.

In closed type medical procedures, an insufflator is arranged to deliver gases to a body cavity of the patient to inflate the body cavity and/or to resist collapse of the body cavity during the medical procedure. Examples of such medical procedures include laparoscopy and endoscopy, although an insufflator may be used with any other type of medical procedure as required. Endoscopic procedures enable a medical practitioner to visualize a body cavity by inserting an endoscope or the like through one or more natural openings, small puncture(s), or incision(s) to generate an image of the body cavity. In laparoscopy procedures, a medical practitioner typically inserts a surgical instrument through one or more natural openings, small puncture(s), or incision(s) to perform a surgical procedure in the body cavity. In some cases an initial endoscopic procedure may be carried out to assess the body cavity, and then a subsequent laparoscopy carried out to operate on the body cavity. Such procedures are widely used, for example, within the peritoneal cavity, or during a thoracoscopy, colonoscopy, gastroscopy or bronchoscopy.

In open type medical procedures, for example, open surgeries, gases are used to fill a surgical cavity, with excess gases spilling outward from the opening. The gases can also be used to provide a layer of gases over exposed body parts, for example, including internal body parts, where there is no discernible cavity. For these procedures, rather than serving to inflate a cavity, the gases can be used to prevent or reduce desiccation and infection by covering exposed internal body parts with a layer of heated, humidified, sterile gases.

An apparatus for delivering gases during these medical procedures can include an insufflator arranged to be connected to a remote source of pressurized gases, for example, a gases supply system in a hospital. The apparatus can be operative to control the pressure and/or flow of the gases from the gases source to a level suitable for delivery into the body cavity, usually via a cannula or needle connected to the apparatus and inserted into the body cavity, or via a diffuser arranged to diffuse gases over and into the wound or surgical cavity.

The internal body temperature of a human patient is typically around 37° C. It can be desirable to match the temperature of the gases delivered from the apparatus as closely as possible to the typical human body temperature. It can also be desirable to deliver gases above or below internal body temperature, such as, for example, 1° C., 2° C., 3° C., 4° C., 5° C., 6° C., 7° C., 8° C., 9° C., 10° C., or 15° C., or more or less above or below internal body temperature for example, or ranges including any two of the foregoing values. It can also be desirable to deliver gases of at a desired fixed or variable humidity and/or a desired fixed or variable gas temperature. The gases at the desired gas temperature and/or humidity (which may be also referred to herein as standard) can be dry cold gas, dry hot gas, humidified cold gas, or humidified hot gas for example. Further, the gases delivered into the patient's body can be relatively dry, which can cause damage to the body cavity, including cell death or adhesions. In many cases, a humidifier is operatively coupled to the insufflator. A controller of the apparatus can energize a heater of the humidifier located in the gases flow path to deliver a humidification fluid to the gases stream prior to entering the patient's body cavity. The humidification fluid may be water vapor.

The humidified gas can be delivered to the patient via further tubing which may also be heated. The insufflator and humidifier can be located in separate housings that are connected together via suitable tubing and/or electrical connections, or located in a common housing arranged to be connected to a remote gas supply via suitable tubing.

### SUMMARY

Condensation can occur on various surfaces on a medical instrument. When condensation forms on a viewing surface of a medical instrument, this is observed as a fogging effect which manifests as an impairment of visibility through a lens or any other viewing surface of a medical instrument (such as, for example, a mirror or transparent or translucent window). When condensation forms on various surfaces of a medical instrument, the condensation can coalesce into water droplets. This can occur directly on the viewing surface or other surfaces, which can then migrate to or be deposited on the viewing surface. Accordingly, as used herein condensation and/or fogging means condensation generally and in some instances, specifically with respect to condensation on a viewing surface (i.e. fogging). Condensation and/or fogging occurs when the temperature of a gas falls below the dew point temperature for the level of humidity the gas is carrying, and/or if there are surfaces significantly below the dew-point temperature. The human body is a warm and humid environment, having a temperature of about 37° C. When cold (for example, at or below typical room temperature and/or below a typical human body temperature) cameras or other medical instruments, are inserted into this environment, condensation can form as fog on the lens, and/or as droplets of water on the scope, which can drip down onto the lens area. The medical instruments may be surgical instruments. Further condensation can also form on the internal wall of the cannula upper housing and drip down, for example, onto the lens area. Further, although the humidification and heating of the insufflation gases can reduce damage to the patient's tissue in the surgical cavity, the humidification and heating of the gases can exacerbate the problem of condensation and/or fogging. Condensation can also occur without external heat or humidification. For example, condensation can result from the inherent temperature (body heat) and humidity (body moisture) of the surgical cavity, and/or from the temperature and humidity of an insufflation fluid.

The fog and/or droplets can impede vision, for example, vision of a surgeon or other medical personnel participating in the medical procedure (for example, surgery). When fogging and/or condensation occurs, it may be necessary to remove the camera and/or the other medical instruments and wipe it (or them) down to remove the fog and/or droplets. However, removing the medical instruments out of the surgical cavity can cause them to cool down again to below the patient's body temperature. As a result, the fogging and/or condensation problem can recur in the absence of any other interventions, for example, pre-warming of the medical instruments, and/or using the light at the end of the camera to warm up the lens. These interventions require additional products and/or expensive surgical systems. The medical instrument may be a surgical instrument.

The present disclosure provides examples of a cannula with heating (for example, using integrated heating elements or removable heating elements) that can remedy the aforementioned problems and/or other problems (including, for example, preventing or at least reducing condensation and/or fogging).

The heated cannula examples disclosed herein can heat, for example, the gases entering the cannula, the instrument, and/or the surgical cavity environment.

In some configurations, the gases can optionally be heated before entering the cannula by a humidifier. The humidifier can reduce cell damage, reduce cell desiccation and can help in reducing post-operative complications, for example, adhesions and/or the like.

In some configurations, the heating cannula described herein can help in reducing fogging and/or condensation by raising the dew point.

In some configurations, portions of the medical instrument may also be heated while introducing the medical instrument back into the surgical cavity to raise the temperature of the medical instrument above the dew point. In some configurations, the medical instrument may be a surgical instrument.

In some configurations, the cannula may include additional elements for example, a venting passage that is configured to vent smoke and/or other gases from the surgical cavity.

In some configurations, a heating element may also be disposed on, within, or around the venting passage to heat the vented gases to prevent condensation and/or fogging within the venting passage.

In some configurations, the heating elements may also be configured to heat a filter located within or adjacent the cannula. The filter may be located within a gases inlet path. The filter can filter gases being delivered to the surgical cavity. Alternatively or additionally, a filter may also be located within the venting passage prior to a vent opening. The filter can filter out smoke and/or odor in the venting gases prior to discharging the venting gases into ambient air.

In some configurations, the heating element(s) disclosed herein can be configured to heat the filter in the inlet and/or the filter in the venting passage.

In some configurations, the cannula may include a single heater that is arranged in contact with the inlet filter and venting filter to heat both simultaneously.

Alternatively, the cannula may include a plurality of heating elements, with at least one associated with the inlet filter and another associated with the venting filter.

In some configurations, the heating elements may be independently controlled to independently heat the inlet filter and venting filter.

In certain examples, the heating element associated with the venting filter may be activated during venting or when a vent is opened. The heater associated with the inlet filter may be activated when gases are flowing into the surgical cavity.

In some configurations, a controller configured to control the heater or heating elements may be the controller in the humidifier or a separate or independent controller for the heater or heating elements.

In some configurations, the heater or heating elements disclosed herein can be incorporated into an insufflation cannula configured to deliver insufflation gases to a surgical cavity, a venting cannula configured to venting the gases from the surgical cavity, and/or a cannula with both a gases delivery passage and a venting passage.

In some configurations, the cannula may also include retaining features that retain the medical instrument in a substantially concentric arrangement or at least restrict radial movement of the medical instrument within the cannula (for example, within a delivery passage of the cannula). The retainer arrangement can assist in ensuring gases flow around the medical instrument when the medical instrument is inserted in the cannula. As disclosed herein, the gases delivered around the medical instrument and/or within the cannula may be warmed by the heater in the cannula.

In some configurations, the cannula may also include one or more of the seals.

In some configurations, the seals can include a heating element.

In some configurations, the seals can be in contact with a medical instrument inserted into the cannula. In some configurations, the medical instrument may be a surgical instrument.

In some configurations, the heating elements in the seals can heat the instrument to reduce and/or remove fogging on the instrument.

In some configurations, the heating elements can also heat the gases passing through the seals.

In some configurations, the cannula may also include separate lumens for gases delivery and instrument insertion.

In some configurations, the heating element may also be configured to heat the gases in the gases delivery lumen to a temperature higher than the standard insufflation gases temperature. When the heated gases meet the instrument inserted through the instrument lumen, for example, near the outlet of the cannula, the heated gases can absorb the moisture on the instrument.

In some configurations, the cannula may include an upper housing defining an inlet and a shaft extending from the housing.

In some configurations, the shaft may include multiple lumens, a first lumen for carrying insufflation gases delivered to the surgical cavity and a second lumen carrying vented gases and/or smoke away from the surgical cavity.

In some configurations, one or more heating elements can be disposed in each lumen. The one or more heating elements can be configured to heat the delivered gases and the vented gases and/or smoke.

In some configurations, the cannula may include an upper housing, a shaft extending from the housing the shaft defining a lumen, a retaining arrangement disposed within the lumen to retain a medical instrument inserted within the lumen, and a heating element disposed in the lumen or on the retaining arrangement to heat the instrument and/or the insufflation gases in the lumen. In some configurations, the medical instrument may be a surgical instrument.

In some configurations, the shaft may include multiple lumens, a first lumen for carrying insufflation gases delivered to the surgical cavity and a second lumen carrying vented gases and/or smoke away from the surgical cavity.

In some configurations, the lumen configured to deliver insufflation gases can include the retaining arrangement.

In some configurations, one or more heating elements can be disposed in each lumen. The one or more heating elements can be configured to heat the delivered gases and the vented gases and/or smoke.

In some configurations, the heated cannula examples disclosed herein can include a guiding element configured to guide a medical instrument, for example, a scope or another surgical instrument, into the cannula to retain the medical instrument in a substantially concentric orientation. The guiding element can assist in retaining the medical instrument in the cannula such that the medical instrument does not contact the cannula wall so as to allow the medical instrument be surrounded by the gases.

In some configurations, the heating element can be flexible.

In some configurations, the heating element can comprise an arcuate shape.

In some configurations, the heating element can comprise a flexible band.

In some configurations, the heating element can comprise a heater wire. In some configurations, the heater wire can helically extend along the elongate shaft or the cannula upper housing. In some configurations, the heating element can comprise a flexible or rigid PCB. In some configurations, the heating element can comprise a thermo-elastic plastic material. In some configurations, the thermo-elastic plastic material can comprise a planar sheet that is bendable and/or malleable.

The heated cannula examples disclosed herein can prevent condensation and/or fogging through heat radiation and/or conduction causing the medical instruments to heat up, and/or through further heating the insufflation gases to reduce the possibility of the humidified and heated insufflation gases falling below (and/or near) the dew point, maintain gases temperature for improved heated humidity therapy, and/or can eliminate condensation and/or fogging once it has occurred through heat radiation and conduction causing fluid evaporation.

The heated cannula examples disclosed herein can also reduce and/or prevent condensation in any filters that may be attached to the cannula.

The heated cannula examples disclosed herein can be advantageous compared with the current surgical mitigation techniques of completely removing the medical instrument from the cannula to apply anti-fog solution and wiping it because the de-fogging process is more streamlined.

As cannulas are a necessary part of certain surgical procedures, for example, the laparoscopic procedure, the heated cannulas can allow cleaning of the medical instrument, for example, the scope or another surgical instrument, without impacting the surgical performance or adding any extra components or complexities to the procedure.

In some configurations, a surgical cannula for providing insufflation gases to a surgical cavity and providing a passage for insertion of one or more medical instruments can include a cannula upper housing including an opening. The cannula can include an elongate shaft extending from the cannula upper housing. The shaft can define a hollow passage to provide the insufflation gases to the surgical cavity. The passage can also be configured to receive a medical instrument. The cannula can include a heating element disposed on or within at least a portion of the cannula along a longitudinal axis of the cannula. The heating element can be configured to transfer heat to the insufflation gases passing through the cannula and/or a portion of the medical instrument to raise the temperature of the insufflation gases and/or the instrument so as to reduce condensation of the insufflation gases and/or reduce condensation on the medical instrument.

In some configurations, a surgical cannula for providing insufflation gases to a surgical cavity and providing a passage for insertion of one or more medical instruments can include a cannula upper housing including an opening. The cannula can include an elongate shaft extending from the cannula upper housing. The shaft can define a hollow passage to provide the insufflation gases to the surgical cavity. The passage can also be configured to receive a medical instrument. The cannula can include a heating element disposed on or within at least a portion of the cannula along a longitudinal axis of the cannula. The heating element can be configured to transfer heat to the insufflation gases passing through the cannula and/or a portion of the medical instrument to raise the temperature of the insufflation gases and/or the instrument so as to reduce and/or prevent condensation of the insufflation gases and/or reduce and/or prevent condensation and/or fogging on the medical instrument.

In some configurations, the medical instrument may be a surgical instrument.

In some configurations, the heating element can extend at least a partial length of the elongate shaft. In some configurations, the heating element can extend substantially along an entire length of the elongate shaft.

In some configurations, the heating element can be disposed within a wall of the elongate shaft.

In some configurations, the heating element can be disposed closer to an inner surface than to an outer surface of the elongate shaft. In some configurations, the heating element can be disposed closer to an outer surface than to an inner surface of the elongate shaft.

In some configurations, the heating element can be located on an inner surface of a sleeve that circumferentially surrounds at least a portion of the elongate shaft.

In some configurations, a location of the sleeve along the longitudinal axis of the cannula can be variable.

In some configurations, the heating element can extend at least a partial length of the cannula upper housing.

In some configurations, the heating element can be located within a wall of the cannula upper housing.

In some configurations, the heating element can be configured to heat the medical instrument as the medical instrument is removed from the cannula.

In some configurations, the heating element can be isolated from the insufflation gases such that the heating element is out of an insufflation gases flow path.

In some configurations, the heating element can be commensurate with a cross-sectional profile of the hollow passage and/or the opening.

In some configurations, the heating element can extend at least substantially circumferentially around the hollow passage of the elongate shaft or the opening of the cannula upper housing.

In some configurations, the heating element can be flexible.

In some configurations, the heating element can comprise an arcuate shape.

In some configurations, the heating element can comprise a flexible band.

In some configurations, the heating element can comprise a heater wire.

In some configurations, the heater wire can helically extend along the elongate shaft or the cannula upper housing.

In some configurations, the heating element can comprise a flexible or rigid PCB.

In some configurations, the heating element can comprise a thermo-elastic plastic material.

In some configurations, the thermo-elastic plastic material can comprise a planar sheet that is bendable and/or malleable.

In some configurations, the cannula can comprise one or more electrical wires in electrical communication with the heating element. The one or more electrical wires can extend along and/or through a wall of the cannula.

In some configurations, the cannula can comprise an inlet for receiving the insufflation gases. The inlet can be in fluid communication with the opening of the cannula upper housing and/or the hollow passage of the elongate shaft.

In some configurations, the inlet can comprise an electrical connector. The electrical connector can be in electrical communication with the one or more electrical wires. The electrical connector can be configured to couple to a corresponding connector on a gases supply tube to supply power to the heating element via the electrical wires.

In some configurations, the connection between the electrical connector and the corresponding connector can comprise a socket connection.

In some configurations, the heating element can be powered by a controller of a humidifier, an independent controller, or a controller of an insufflator.

In some configurations, the elongate shaft can comprise a second hollow passage.

In some configurations, the cannula can comprise a second heating element extending around the second hollow passage.

In some configurations, the second hollow passage can be offset from the hollow passage and is adjacent a portion of the heating element.

In some configurations, the elongate shaft can comprise a plurality of lumens.

In some configurations, the heating element can be disposed in one lumen or more lumens to heat gases passing through the one or more lumens.

In some configurations, the elongate shaft can comprise two lumens and the heating element can comprise first and second heating elements disposed in both lumens respectively.

In some configurations, the first heating element can heat insufflation gases and the second heating element can heat vented gases and/or smoke.

In some configurations, the cannula can comprise a filter disposed on or within the cannula.

In some configurations, the heating element can be positioned so as to heat the filter.

In some configurations, the heating element can be positioned to contact the filter.

In some configurations, a surgical cannula for providing insufflation gases to a surgical cavity and providing a passage for insertion of one or more medical instruments can include a cannula upper housing including an opening. The cannula can include an elongate shaft extending from the cannula upper housing. The shaft can define a hollow passage to provide the insufflation gases to the surgical cavity. The passage can also be configured to receive a medical instrument. The cannula can include a heating element disposed on or within at least a portion of the cannula along a longitudinal axis of the cannula. The heating element can be configured to increase a temperature of the insufflation gases passing through the cannula and/or the instrument above a dew point to reduce condensation of the gases and/or reduce condensation on the medical instrument.

In some configurations, a surgical cannula for providing insufflation gases to a surgical cavity and providing a passage for insertion of one or more medical instruments can include a cannula upper housing including an opening. The cannula can include an elongate shaft extending from the cannula upper housing. The shaft can define a hollow passage to provide the insufflation gases to the surgical cavity. The passage can also be configured to receive a medical instrument. The cannula can include a heating element disposed on or within at least a portion of the cannula along a longitudinal axis of the cannula. The heating element can be configured to increase a temperature of the insufflation gases passing through the cannula and/or the instrument above a dew point to reduce and/or prevent condensation of the gases and/or reduce and/or prevent condensation and/or fogging on the medical instrument.

In some configurations, the medical instrument may be a surgical instrument.

In some configurations, the heating element can extend at least a partial length of the elongate shaft. In some configurations, the heating element can extend substantially along an entire length of the elongate shaft.

In some configurations, the heating element can be disposed within a wall of the elongate shaft.

In some configurations, the heating element can be disposed closer to an inner surface than to an outer surface of the elongate shaft. In some configurations, the heating element can be disposed closer to an outer surface than to an inner surface of the elongate shaft.

In some configurations, the heating element can be located on an inner surface of a sleeve that circumferentially surrounds at least a portion of the elongate shaft.

In some configurations, a location of the sleeve along the longitudinal axis of the cannula can be variable.

In some configurations, the heating element can extend at least a partial length of the cannula upper housing.

In some configurations, the heating element can be located within a wall of the cannula upper housing.

In some configurations, the heating element can be configured to heat the medical instrument as the medical instrument is removed from the cannula.

In some configurations, the heating element can be isolated from the insufflation gases such that the heating element is out of an insufflation gases flow path.

In some configurations, the heating element can be commensurate with a cross-sectional profile of the hollow passage and/or the opening.

In some configurations, the heating element can extend at least substantially circumferentially around the hollow passage of the elongate shaft or the opening of the cannula upper housing.

In some configurations, the heating element can be flexible.

In some configurations, the heating element can comprise an arcuate shape.

In some configurations, the heating element can comprise a flexible band.

In some configurations, the heating element can comprise a heater wire.

In some configurations, the heater wire can helically extend along the elongate shaft or the cannula upper housing.

In some configurations, the heating element can comprise a flexible or rigid PCB.

In some configurations, the heating element can comprise a thermo-elastic plastic material.

In some configurations, the thermo-elastic plastic material can comprise a planar sheet that is bendable and/or malleable.

In some configurations, the cannula can comprise one or more electrical wires in electrical communication with the heating element. The one or more electrical wires can extend along and/or through a wall of the cannula.

In some configurations, the cannula can comprise an inlet for receiving the insufflation gases. The inlet can be in fluid communication with the opening of the cannula upper housing and/or the hollow passage of the elongate shaft.

In some configurations, the inlet can comprise an electrical connector. The electrical connector can be in electrical communication with the one or more electrical wires. The electrical connector can be configured to couple to a corresponding connector on a gases supply tube to supply power to the heating element via the electrical wires.

In some configurations, the connection between the electrical connector and the corresponding connector can comprise a socket connection.

In some configurations, the heating element can be powered by a controller of a humidifier, an independent controller, or a controller of an insufflator.

In some configurations, the elongate shaft can comprise a second hollow passage.

In some configurations, the cannula can comprise a second heating element extending around the second hollow passage.

In some configurations, the second hollow passage can be offset from the hollow passage and is adjacent a portion of the heating element.

In some configurations, the elongate shaft can comprise a plurality of lumens.

In some configurations, the heating element can be disposed in one lumen or more lumens to heat gases passing through the one or more lumens.

In some configurations, the elongate shaft can comprise two lumens and the heating element can comprise first and second heating elements disposed in both lumens respectively.

In some configurations, the first heating element can heat insufflation gases and the second heating element can heat vented gases and/or smoke.

In some configurations, the cannula can comprise a filter disposed on or within the cannula.

In some configurations, the heating element can be positioned so as to heat the filter.

In some configurations, the heating element can be positioned to contact the filter.

In some configurations, a surgical system for supplying insufflation gases to a surgical cavity can comprise a gases supply configured to provide the insufflation gases. The surgical system may be an insufflation system. The system can comprise a humidifier in fluid communication with the gases supply and configured to humidify the insufflation gases received from the gases supply. The system can comprise any of the surgical cannula disclosed herein. The system can include a gases delivery tube extending between and in fluid communication with the humidifier and the surgical cannula, respectively. The gases delivery tube can be in electrical communication with the humidifier and the surgical cannula, respectively. The gases delivery tube can direct the insufflation gases into the surgical cannula and direct an electrical current from the humidifier to the heating element within the surgical cannula.

In some configurations, the humidifier can be a passover humidifier including a water chamber configured to hold a volume of a humidification fluid. In some configurations, the humidifier can be a passover humidifier including a water chamber configured to hold a volume of water.

In some configurations, the humidification chamber can be in fluid communication with the gases supply such that the insufflation gases are humidified by water vapor wicked from the volume of water.

In some configurations, the humidifier can comprise a heater plate including a heater plate heating element and a humidification chamber positionable on the heater plate.

In some configurations, the humidification chamber can be configured to hold a volume of a humidification fluid that is heated by the heater plate heating element to create water vapor. The humidification chamber can be in fluid communication with the gases supply such that the insufflation gases are humidified by the water vapor. In some configurations, the humidification chamber can be configured to hold a volume of water that is heated by the heater plate heating element to create water vapor. The humidification chamber can be in fluid communication with the gases supply such that the insufflation gases are humidified by the water vapor.

In some configurations, the humidifier can be located outside a sterile zone.

In some configurations, the humidifier can be located adjacent the gases supply.

In some configurations, the gases supply can be an insufflator.

In some configurations, the insufflator can be configured to provide a continuous or intermittent flow of gases.

In some configurations, the gases delivery tube can be a spirally wound tube.

In some configurations, an electrical circuit can be located within walls of the gases delivery tube.

In some configurations, the surgical cannula can comprise a filter module that is removably coupled to or integrated with the surgical cannula.

In some configurations, the heating element of the surgical cannula can be arranged in contact with or extends through the filter module.

In some configurations, a method of reducing condensation on a medical instrument within a body cavity can comprise inserting a cannula into the body cavity; inserting a medical instrument through a channel of the cannula; flowing insufflation gases through the cannula; and heating the insufflation gases directly adjacent the medical instrument to above a predetermined dew point sufficiently to prevent or reduce condensation on the medical instrument.

In some configurations, a method of reducing condensation and/or fogging on a medical instrument within a body cavity can comprise inserting a cannula into the body cavity; inserting a medical instrument through a channel of the cannula; flowing insufflation gases through the cannula; and heating the insufflation gases directly adjacent the medical instrument to above a predetermined dew point sufficiently to prevent or reduce condensation and/or fogging on the medical instrument.

In some configurations, the medical instrument may be a surgical instrument.

In some configurations, the medical instrument can comprise an optical element, and heating the insufflation gases can be sufficient to prevent or reduce condensation and/or fogging on the optical element.

In some configurations, the method can further comprise measuring the temperature proximate the optical element, and adjusting the heating of the insufflation gases sufficiently to maintain the temperature proximate the optical element above the predetermined dew point.

In some configurations, a surgical cannula for providing insufflation gases into a surgical cavity and for receiving a surgical instrument into the surgical cavity can comprise an elongated outer tubular member including opposing proximal and distal end portions and a longitudinal axis extending therethrough; an elongated inner tubular member including opposing proximal and distal end portions and being arranged coaxially within the outer tubular member, the inner tubular member defining a central lumen for introduction of the surgical instrument therethrough; an insufflation passage defined between an outer surface of the inner tubular member and an inner surface of the outer tubular member, the insufflation passage in communication with a source of insufflation gas via an inlet of the insufflation gas; and a plurality of apertures extending through a wall of at least the distal portion of the outer tubular member and in fluid communication with the insufflation passage, the plurality of apertures defining an outlet of the insufflation gas from the insufflation passage into the surgical cavity, wherein a heating element can be disposed generally parallel to the longitudinal axis between an outer surface of the outer tubular member and an inner surface of the inner tubular member.

In some configurations, the plurality of apertures can define the sole outlet of the insufflation gas from the insufflation passage into the surgical cavity.

In some configurations, the plurality of apertures can be configured to allow the insufflation gas to be discharged laterally or obliquely relative to the insufflation passage.

In some configurations, the heating element can be embedded in the wall of the outer tubular member or the wall of the inner tubular member.

In some configurations, the heating element can be located within the insufflation passage.

In some configurations, the central lumen can be configured to recirculate gas and/or smoke inside the surgical cavity so as to seal the surgical cavity from ambient air.

In some configurations, the gas and/or smoke inside the surgical cavity recirculating into the central lumen can be configured to provide an air barrier to the ambient air.

In some configurations, the heating element can be powered by battery, a power plug, or a gas delivery tube coupled to the inlet of the insufflation gas.

In some configurations, a surgical cannula for providing insufflation gases into a surgical cavity and for receiving a surgical instrument into the surgical cavity can comprise an outer body located at a proximal end of the cannula; an outer elongate shaft extending distally from the outer body; an inner body located at a proximal end of the cannula; an inner elongate shaft extending distally from the inner body, the inner body and the inner elongate shaft arranged coaxially within the outer body and the outer elongate shaft, the inner body and the inner elongate shaft defining a central lumen for introduction of the surgical instrument therethrough; an insufflation passage defined between the outer surface of the inner body and the inner elongate shaft and the inner surface of the outer body and outer elongate shaft, the insufflation passage in communication with a source of insufflation gas via an inlet of the insufflation gas; and a plurality of apertures extending through a wall of at least the distal portion of the outer elongate shaft and in fluid communication with the insufflation passage, the plurality of apertures defining an outlet of the insufflation gas from the insufflation passage into the surgical cavity, wherein a heating element is disposed generally parallel to a longitudinal axis of the cannula between an outer surface of the outer elongate shaft and/or the outer body and an inner surface of the inner elongate shaft and/or the inner body.

In some configurations, the plurality of apertures can define the sole outlet of the insufflation gas from the insufflation passage into the surgical cavity.

In some configurations, the plurality of apertures can be configured to allow the insufflation gas to be discharged laterally or obliquely relative to the insufflation passage.

In some configurations, the heating element can be embedded in the wall of the outer elongate shaft and/or the outer body or the wall of the inner elongate shaft and/or the inner body.

In some configurations, the heating element can be located within the insufflation passage.

In some configurations, the central lumen can be configured to recirculate gas and/or smoke inside the surgical cavity so as to seal the surgical cavity from ambient air.

In some configurations, the gas and/or smoke inside the surgical cavity recirculating into the central lumen can be configured to provide an air barrier to the ambient air.

In some configurations, the heating element can be powered by battery, a power plug, or a gas delivery tube coupled to the inlet of the insufflation gas.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure. In some cases, a “slice” has been shown for clarity purposes for some sectional and cross-sectional views of a three dimensional cannula. A person reasonably skilled in the art would be able to appreciate from the disclosure herein that these figures illustrate a slice of a three dimensional cannula. Certain features may not be shown in the slices, for example, any projected surfaces including but not limited to hole surface projections. A person reasonably skilled in the art would be able to appreciate from the disclosure herein that the three dimensional cannula with such slices can include those features.

FIG. 1 illustrates schematically an example medical gases delivery apparatus in use in surgery.

FIGS. 2A-2C illustrate schematically example medical gases delivery apparatuses in use in surgery.

FIGS. 3A-3B illustrate perspective and partial front views of a cannula with a shaft heater sectioned along a central longitudinal plane.

FIG. 3C illustrates a transverse cross-sectional view of the cannula of FIG. 3A.

FIG. 4A illustrates a partial longitudinal cross-sectional view of a cannula having double concentric lumens.

FIG. 4B illustrates a transverse cross-sectional view of the cannula of FIG. 4A.

FIG. 5A illustrates a partial longitudinal cross-sectional view of a cannula having double offset lumens.

FIG. 5B illustrates a transverse cross-sectional view of the cannula of FIG. 5A.

FIGS. 6A-6B illustrate perspective and partial front views of another cannula with a heater associated with a portion of the cannula shaft sectioned along a central longitudinal plane.

FIG. 6C illustrates a transverse cross-sectional view of the cannula of FIG. 6A.

FIGS. 7A-7B illustrate perspective and partial front views of another cannula with a heater associated with a portion of the cannula shaft sectioned along a central longitudinal plane.

FIG. 7C illustrates a transverse cross-sectional view of the entire cannula of FIG. 7A.

FIGS. 8A-8B illustrate perspective and partial longitudinal cross-sectional views of a cannula with a heater sleeve.

FIG. 8C illustrates a transverse cross-sectional view of the cannula of FIG. 8A.

FIGS. 9A-9B illustrate perspective and partial front views of a cannula with a body heater sectioned along a central longitudinal plane.

FIG. 9C illustrates a transverse cross-sectional view of the cannula of FIG. 9A.

FIGS. 10A-10H illustrate various examples of heated seal(s) in a cannula.

FIGS. 11A-11C illustrate a heated gases flow in a concentric multi-lumen cannula.

FIGS. 12A-12D illustrate examples of heating elements in the cannula.

FIGS. 13A-13B illustrate disconnected and connected configurations of an additional example of power supply to the heating element(s).

FIGS. 14A-14C illustrate schematically power source options for heating elements in the cannula.

FIGS. 15A-15C illustrate schematically various heating effect options of a heated cannula.

FIGS. 16A-16D illustrate schematically cross-sectional views of a heated cannula that is sealed pneumatically.

### DETAILED DESCRIPTION

Although certain embodiments and examples are described below, those of skill in the art will appreciate that the disclosure extends beyond the specifically disclosed embodiments and/or uses, and obvious modifications and equivalents thereof. Thus, it is intended that the scope of the disclosure herein disclosed should not be limited by any particular embodiments described below.

### Example Medical Gases Delivery Systems

Fluids, for example, gases, can be introduced to a surgical cavity, for example, the peritoneal cavity via a cannula inserted through an incision made in a patient's body (for example, the abdominal wall). The cannula can be coupled to an insufflator. The gases flow from the insufflator can be increased to inflate the surgical cavity (for example, to maintain a pneumoperitoneum, which is a cavity filled with gas within the abdomen). The introduced gases can inflate the surgical cavity. A medical instrument can be inserted through the cannula into the inflated surgical cavity. The medical instrument may be a surgical instrument. For example, an endoscope, another vision system, including but not limited to, a scope or a camera unit, can be inserted into the cavity and visibility in the cavity can be assisted by insertion of gases, which can be air and/or other fluid, for example, carbon dioxide. After initial insufflation and insertion of the instrument (for example, a laparoscope) through the cannula, additional cannulas can be placed in the surgical cavity under laparoscopic observation. At the end of the operating procedure, all instruments and cannulas are removed from the surgical cavity, the gases are expelled, and each incision is closed. For thoracoscopy, colonoscopy, sigmoidoscopy, gastroscopy, bronchoscopy, and/or others, the same or substantially similar procedure for introducing gases to a surgical cavity can be followed. The quantity and flow of gases can be controlled by the clinician performing the examination and/or automatically by the surgical system. The surgical system can be an insufflation system.

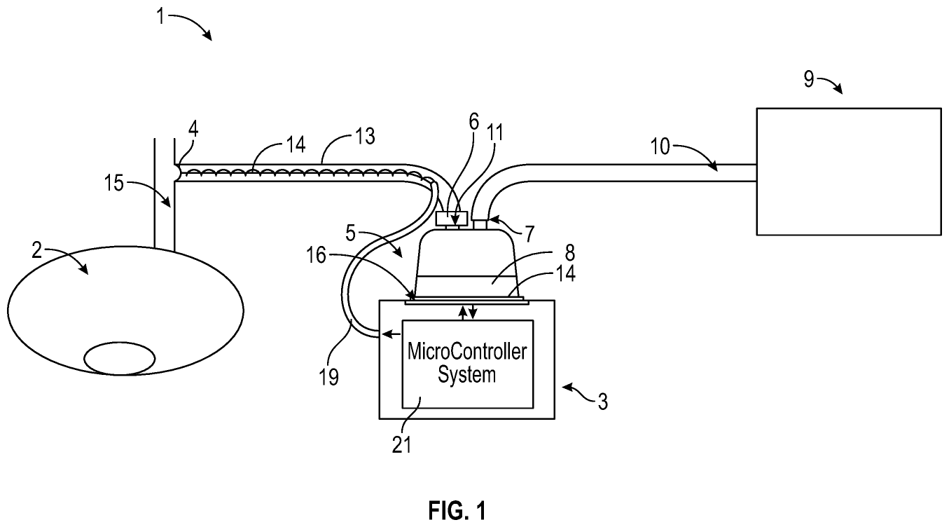

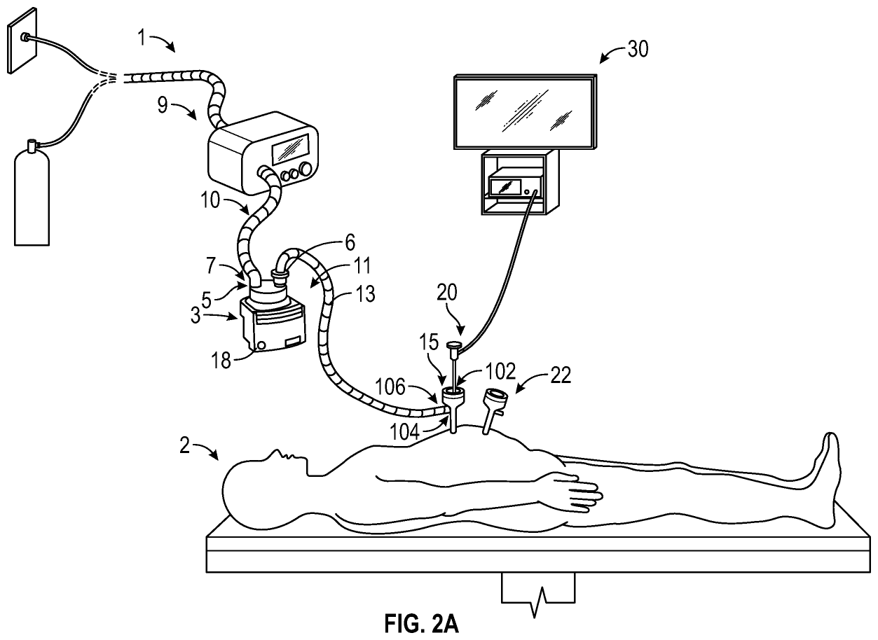

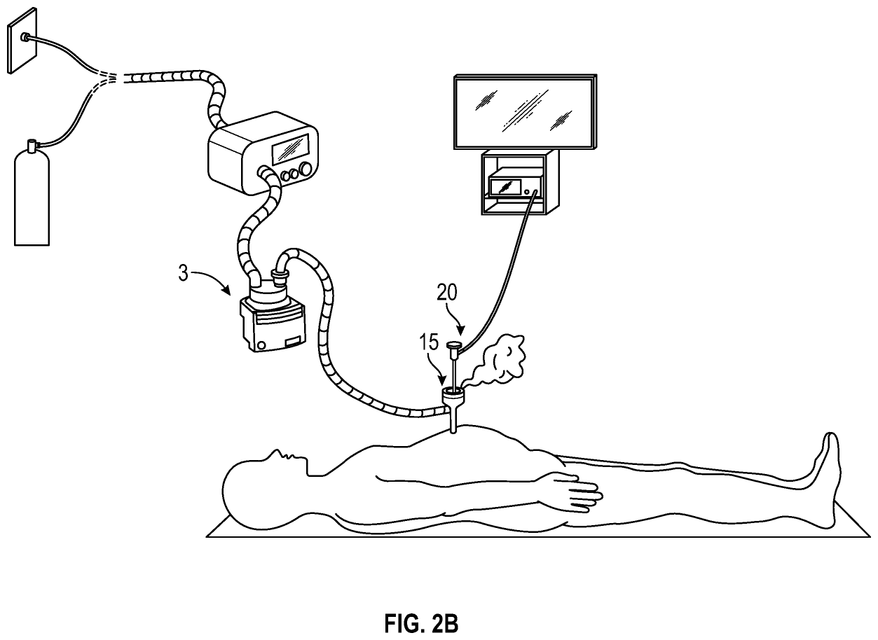

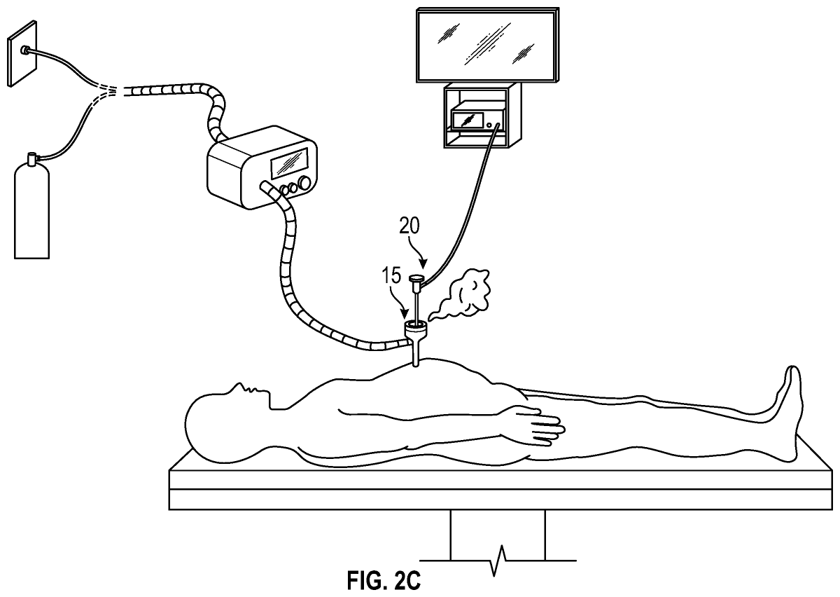

FIGS. 1 and 2A-2B illustrate schematically using an example surgical system 1 during a medical procedure. Features of FIGS. 1 and 2A-2B can be incorporated into each other. The same features have the same reference numerals in FIGS. 1 and 2A-2B. As shown in FIG. 1, the patient 2 can have a cannula 15 inserted within a cavity of the patient 2 (for example, an abdomen of the patient 2 in the case of a laparoscopic surgery), as previously described.

As shown in FIGS. 1 and 2A-2B, the cannula 15 can be connected to a gases delivery conduit 13 (for example, via a Luer lock connector 4). The cannula 15 can be used to deliver gases into a surgical site, for example, within the cavity of the patient 2. The cannula 15 can include one or more passages to introduce gases and/or one or more surgical instruments 20 into the surgical cavity. The surgical instrument can be a scope, a tool for electrocautery, electro-surgery, energy and laser cutting and/or cauterizing, among others, or any other instrument. The surgical instrument 20 can be coupled to an imaging device 30, which can have a screen. The imaging device 30 can be part of a surgical system. The surgical system may be a surgical stack.

As shown in FIG. 2A, the system can also optionally include a venting cannula 22, which can have substantially the same features as the cannula 15. The venting cannula may include a valve that allows venting. The valve can be automatically controlled by a controller associated with the gases source (i.e. insufflator), by a controller in the humidifier, or by an independent controller of the system. The valve can also be manually actuated (for example, by turning a tap by hand or by a foot pedal, or otherwise). The venting cannula 22 can be coupled to a filtration system to filter out smoke and the like. The venting cannula 22 can also alternatively be coupled to a recirculation system that is configured to recirculate the gases from the surgical cavity back to the insufflator for re-delivery into the surgical cavity. The gases can be filtered and/or dehumidified prior to being returned to the insufflator. In certain configurations, the cannula 15 may include two or more passages. One passage can be configured to deliver gases and/or the medical instrument into the surgical cavity. Another passage can be configured to vent gases out of the surgical cavity. The venting passage may include a valve and/or passive vent openings. The cannula 15 may also include a retaining arrangement (for example, ribs and/or the like) to retain the medical instrument (for example, a scope or another surgical instrument) in a substantially concentric orientation relative to the delivery passage. As shown in FIGS. 2B and 2C, the same cannula 15 can be used for both gases delivery and venting.

The gases delivery conduit 13 can be made of a flexible plastic and can be connected to a humidifier chamber 5. The humidifier chamber 5 can optionally or preferably be in serial connection to a gases supply 9 via a further conduit 10. The gases supply or gases source can be an insufflator, bottled gases, or a wall gases source. The gases supply 9 can provide the gases without humidification and/or heating. A filter 6 be connected downstream of the humidifier's outlet 11. The filter can also be located along the further conduit 10, or at an inlet of the cannula 15. The filter can be configured to filter out pathogens and particulate matter in order to reduce infection or contamination of the surgical site from the humidifier or gases source. The gases supply can provide a continuous or intermittent flow of gases. The further conduit 10 can also preferably be made of flexible plastic tubing.

The gases supply 9 can provide one or more insufflation fluid including liquid and/or gases, for example, carbon dioxide, to the humidifier chamber 5. The gases supply can provide a continuous gases flow or an intermittent gases flow. The gases can be humidified as they are passed through the humidifier chamber 5, which can contain a volume of water or any other type of humidification fluid 8. As shown in FIG. 2C, the gases supply can also be directly connected to the cannula 15 without a humidifier unit. The gases can be dry cold gas, dry hot gas, humidified gas, or otherwise. Optionally, the gases supply 9 can include two gas sources.

A humidifier that incorporates the humidifier chamber 5 can be any type of humidifier. The humidifier chamber 5 can include plastic formed chamber having a metal or otherwise conductive base 14 sealed thereto. The base can be in contact with the heater plate 16 during use. The volume of water 8 contained in the chamber 5 can be heated by a heater plate 16, which can be under the control of a controller or control means 21 of the humidifier. The volume of water 8 within the chamber 5 can be heated such that it evaporates, mixing water vapor with the gases flowing through the chamber 5 to heat and humidify the gases.

The controller or control means 21 can be housed in a humidifier base unit 3, which can also house the heater plate 16. The heater plate 16 can have an electric heating element therein or in thermal contact therewith. Optionally one or more insulation layers can be located between in the heater plate 16 and the heater element. The heater element can be a base element (or a former) with a wire wound around the base element. The wire can be a nichrome wire (or a nickel-chrome wire). The heater element can also include a multi-layer substrate with heating tracks electrodeposited thereon or etched therein. The controller or control means 21 can include electronic circuitry, which can include a microprocessor for controlling the supply of energy to the heating element. The humidifier base unit 3 and/or the heater plate 16 can be removably engageable with the humidifier chamber 5. The humidifier chamber 5 can also alternatively or additionally include an integral heater. Alternatively, the controller or control means 21 can be housed or partially housed external to the humidifier base unit 3.

The heater plate 16 can include a temperature sensor, for example, a temperature transducer or otherwise, which can be in electrical connection with the controller 21. The heater plate temperature sensor can be located within the humidifier base unit 3. The controller 21 can monitor the temperature of the heater plate 16, which can approximate a temperature of the water 8.

A temperature sensor can also be located at the or near the outlet 11 to monitor a temperature of the humidified gases leaving the humidifier chamber 5 from the outlet 11. The temperature sensor can also be connected to the controller 21 (for example, with a cable or wirelessly). Additional sensors can also optionally be incorporated, for example, for sensing characteristics of the gases (for example, temperature, humidity, flow, or others) at a patient end of the gases delivery conduit 13.

The gases can exit out through the humidifier's outlet 11 and into the gases delivery conduit 13. The gases can move through the gases delivery conduit 13 into the surgical cavity of the patient 2 via the cannula 15, thereby inflating and maintaining the pressure within the cavity. The gases delivery conduit 13 can be made of plastic or other suitable materials. Preferably, the gases leaving the outlet 11 of the humidifier chamber 5 can have a relative humidity of up to 100%, for example, at around 100%. As the gases travel along the gases delivery conduit 13, further condensation can occur so that water vapor can condense on a wall of the gases delivery conduit 13. Further condensation can have undesirable effects, for example, detrimentally reducing the water content of the gases delivered to the patient. In order to reduce and/or minimize the occurrence of condensation within the gases delivery conduit 13, a heater wire 14 can be provided within, throughout, or around the gases delivery conduit 13. The heater wire 14 can be electronically connected to the humidifier base unit 3, for example by an electrical cable 19 to power the heater wire.

The heater wire 14 can include an insulated copper alloy resistance wire, other types of resistance wire, or other heater element, and/or be made of any other appropriate material. The heater wire can be a straight wire or a helically wound element. An electrical circuit including the heater wire 14 can be located within walls of the gases delivery tube 13. The gases delivery tube 13 can be a spiral wound tube. The heater wire 14 can be spirally wound around an insulating core of the gases delivery conduit 13. The insulating coating around the heater wire 14 can include a thermoplastics material which, when heated to a predetermined temperature, can enter a state in which its shape can be altered and the new shape can be substantially elastically retained upon cooling. The heater wire 14 can be wound in a single or double helix. Measurements by the temperature sensor and/or the additional sensor(s) at the patient end of the conduit 13 can provide feedback to the controller 21 so that the controller 21 can optionally energize the heater wire to increases and/or maintain the temperature of the gases within the gases delivery conduit 13 (for example, above or below internal body temperature of about 37° C., such as, for example, 1° C., 2° C., 3° C., 4° C., 5° C., 6° C., 7° C., 8° C., 9° C., 10° C., or 15° C. or more or less above or below internal body temperature for example, or ranges including any two of the foregoing values).

The controller or control means 21 can, for example, include the microprocessor or logic circuit with associated memory or storage means, which can hold a software program. When executed by the control means 21, the software can control the operation of the surgical system 1 in accordance with instructions set in the software and/or in response to external inputs. The surgical system may be an insufflation system. For example, the controller or control means 21 can be provided with input from the heater plate 16 so that the controller or control means 21 can be provided with information on the temperature and/or power usage of the heater plate 16. The controller or control means 21 can be provided with inputs of temperature of the gases flow. For example, the temperature sensor can provide input to indicate the temperature of the humidified gases flow as the gases leave the outlet 11 of the humidifier chamber 5. A flow sensor can also be provided in the same position as or near the temperature sensor or at other appropriate location within the surgical system 1. The controller 21 can control a flow controller which regulates the flow rate of gases through the system 1. The flow controller may be a flow regulator. The flow controller can include a flow inducer and/or inhibiter such as a motorized fan for example. Valves and/or vents can additionally or alternatively be used to control the gases flow rate.

A patient input 18 located on the humidifier base unit 3 can allow a user (for example, a surgeon or nurse) to set a desired gases temperature and/or gases humidity level to be delivered. Other functions can also optionally be controlled by the user input 18, for example, control of the heating delivered by the heater wire 14. The controller 21 can control the system 1, and in particular to control the flow rate, temperature, and/or humidity of gas delivered to the patient, to be appropriate for the type of medical procedure for which the system 1 is being used.

The humidifier base unit 3 can also include a display for displaying to the user the characteristics of the gas flow being delivered to the patient 2.

Although not shown, the humidifier can also optionally be a passover or bypass humidifier, which can include the chamber with a volume of water or any other type of humidification fluid, but may not include a heater plate for heating the humidification fluid. The chamber can be in fluid communication with the gases supply such that the insufflation gases are humidified by humidification fluid wicked from the volume of water as the insufflation gases pass over the volume of humidification fluid.

When in use, the humidifiers described above can be located outside an operating sterile zone and/or adjacent the insufflator. As a result, the medical personnel would not be required to touch the humidifier when moving the cannula during the operation to maneuver the medical instruments within the surgical cavity. The medical instruments may include a surgical instrument. The humidifier may not need to be sterilized to the same extent as the medical instruments. Furthermore, the humidifier being located outside the operating sterile zone can reduce obstructions to the medical personnel during the operating procedure that may restrict movements of the medical personnel and/or the medical instruments in the already crowded space.

### Examples of Heated Cannulas

Condensation and/or fogging occurs when the temperature of a gas falls below the dew point temperature for the level of humidity the gas is carrying, and/or if there are surfaces significantly below the dew-point temperature. In FIGS. 1 and 2, as the insufflation gases travel from the gas delivery tube 13 into the cannula 15, the heated and humidified gases can cool down to be closer to the dew point within the cannula 15 if the cannula 15 is not heated. Further, as described above, one or more medical instruments, for example, cameras, surgical scopes and/or another surgical instrument, which are at a temperature lower than the human body, can be inserted into the surgical cavity via the cannula 15. The humidified gases can thus condense as fog on the lens, and/or as droplets of water on the surgical scope, which can drip down onto the lens area. The fog and/or droplets can impede vision, for example, vision of a surgeon or other medical personnel participating in the surgery. Removal of the medical instruments to wipe off the fog and/or droplets can slow down the surgical procedure and/or result in further condensation and/or fogging re-occurring upon reinserting the medical instruments, which would have cooled down when removed from the surgical cavity.

The present disclosure provides examples of a cannula, which can be used as the cannula 15 disclosed herein, and which includes integrated heating for reducing, preventing, and/or removing condensation and/or fogging of the medical instruments without requiring additional components or tools. The medical instruments may include a surgical instrument. The cannula can be single use (disposable) or reusable. Alternatively, parts of the cannula can be single use (disposable) or reusable. The cannula may be made of materials that are biocompatible and/or sterilizable. In the present disclosure, features of the different examples of heated cannulas can be incorporated into or combined with one another.

The example heated cannulas disclosed herein can be implemented into existing surgical systems without requiring customized and/or more expensive surgical systems. The surgical systems can be insufflation systems. The example heated cannulas disclosed herein can therefore improve optical clarity of the camera lens and/or maintain a clear field of vision, which can aid in minimizing operation time and post-operation pain and/or complication, and/or can make it easier for the medical personnel, for example, the surgeon, in navigating the cannula in the surgical cavity during the medical procedure. Heating of the cannula can also allow better control of the insufflation therapy, for example, by increasing or maintaining a temperature and/or humidity of the gases delivered to the patient. Heat transferred to the cannula can be transmitted to the medical instrument inserted into the cannula via the heated gases. Heating the gases flow, for example, by using the heating element examples disclosed herein, can thus maintain therapy temperature of the insufflation gases and/or therapy conditions, reduce and/or prevent condensation in the venting path, reduce and/or prevent condensation in the delivery passage, and/or maintain effectiveness of the therapy. The medical instrument may be a surgical instrument.

The example heated cannulas can have any of features of the cannula 15 described above. For example, the heated cannula can have a cannula upper housing 102 connected to an elongate shaft 104. The cannula upper housing 102 can house one or more instrument seals. The upper housing 102 can define an opening. The elongate shaft 104 can optionally have a pointed end such that the cannula can function as a trocar for easier insertion of the cannula 15 into the surgical cavity. A trocar can include a cannula and an obturator. The cannula upper housing 102 can have a greater cross-sectional dimension than the elongate shaft 104 for easier insertion of the medical instruments. The medical instruments may include a surgical instrument. As shown in FIG. 2A, the cannula upper housing 102 can have generally a funnel shape, with a cross-sectional dimension (for example, diameter) decreasing from a location further from the elongate shaft 104 to a location closer to the elongate shaft 104. A gases inlet 106 can be located on the cannula upper housing 102. The cannula upper housing 102 can include a cavity. The elongate shaft 104 can include a hollow passage. The cavity and the hollow passage can be in fluid communication. The heated cannula can include a heating element releasably coupled to (for example, via a sleeve) or integrated into the heated cannula (for example, in at least a portion of the cannula upper housing 102 and/or a portion of the elongate shaft 104). The heated cannula can include a filter module that is removably coupled to or integrated with the cannula (for example, located proximally in the cannula upper housing 102 or a sleeve attached (described below), or coupled to the cannula via a tube). The heating element can be arranged to be in contact with or extends through the filter module.

A surgical system for supplying insufflation gases to a surgical cavity, for example, any surgical systems disclosed above (which can include insufflation systems), can incorporate any of the example heated cannulas disclosed herein. As described above, the system can include a gases supply configured to provide the insufflation gases, a humidifier in fluid communication with the gases supply and configured to humidify the insufflation gases received from the gases supply, and a gases delivery tube extending between and in fluid communication with the humidifier and the cannula, respectively. The gases delivery tube can also be in electrical communication with the humidifier and the cannula, respectively. When the system is in use, the gases delivery tube can direct the insufflation gases into the surgical cannula and can also direct an electrical current from the humidifier to the heating element within the cannula. The heating element can be configured to transfer heat to the insufflation gases passing through the cannula, and/or a portion of the medical instrument inserted into and/or removed from the cannula, to raise the temperature of the gases and/or the instrument so as to reduce and/or prevent condensation and/or fogging. The temperature of the insufflation gases and/or the instrument can be increased above a dew point to prevent condensation of the gases and/or reduce and/or prevent condensation and/or fogging (and/or remove by evaporation condensation and/or fogging already formed) on the medical instrument. The temperature of the insufflation gases and/or the instrument (for example, on or near an optical elements, for example, a camera lens, or other area of the instrument) can also be measured, for example, with thermocouples and/or other sensors, and closed loop feedback can be provided to a controller to maintain the temperature proximate the instrument at or above a predetermined or calculated value, for example, the dew point. The medical instrument may be a surgical instrument.

The heating elements disclosed herein can also be installed in a venting cannula (for example, the venting cannula 22) configured to vent the gases and/or smoke from the surgical cavity. Heating the vented gases and/or a filter in the venting cannula can reduce and/or prevent condensation and/or clogging in the venting filter.

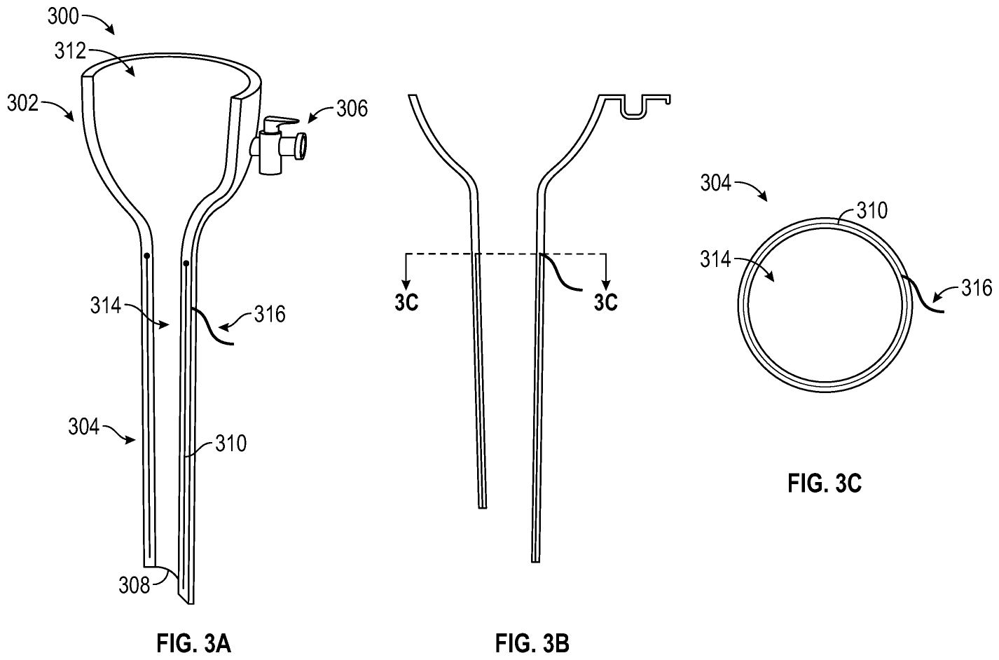

More detailed examples of the heating element are described below with reference to FIGS. 3A-11B. As described herein, a proximal direction with respect to a medical instrument generally can refer to the top end of the medical instrument body, while a distal direction with respect to a medical instrument generally can refer to the bottom end of the medical instrument body configured to be the first section of the medical instrument inserted into the cannula and/or surgical cavity. Reference numerals of the same or substantially the same features share the same last two digits.

### Examples of a Heater Associated with a Cannula Shaft

FIGS. 3A-8C illustrate examples of a cannula with the heating element located along the elongate shaft. FIGS. 3A, 6A, and 7A show perspective views of a cannula 300, 600, 700 sectioned along a central longitudinal plane to better illustrate the heating element 310, 610, 710.

As shown in FIGS. 3A-3C, the cannula 300 can include a cannula upper housing 302 and an elongate shaft 304 extending from the cannula upper housing 302. A free end 308 of the elongate shaft 304 can optionally have a pointed or otherwise sharp end 308, or a square tip. The cannula upper housing 302 can have a cavity 312, which can be in fluid communication with (for example, being connected to or continuous with) a hollow passage 314 of the elongate shaft 304. The cannula 300 can include a gases inlet 306 coupled to a wall of the cannula upper housing 302. The gases inlet 306 can be connected to a gases delivery tube of an surgical system (for example, an insufflation system, and/or any of the systems disclosed herein). As shown in FIG. 3B, the inlet 306 can be in fluid communication with the cavity 312 of the cannula upper housing 302 and/or the hollow passage 314 of the elongate shaft 304.

A heating element 310 can be embedded in a wall of the elongate shaft 304. The heating element 310 can be moulded into the wall of the elongate shaft 304. As described in the present disclosure, when moulding a component, for example, the heating element, into the wall of the elongate shaft, the component can be at an edge of the wall of the elongate shaft or embedded within the wall of the elongate shaft wall. As shown in FIGS. 3A and 3B, the heating element 310 can extend substantially along an entire length of the elongate shaft 310. The heating element 310 can be flexible. As shown in FIG. 3C, the heating element 310 can be commensurate with a cross-sectional profile of the elongate shaft 304. The heating element 310 can extend circumferentially or at least substantially circumferentially around the hollow passage 314 of the elongate shaft 304. The heating element 310 may also extend at least about half of the circumference of the elongate shaft 304. The heating element 310 may also include additional circuits and/or electrical insulators to avoid short circuits or shocking a patient or user. The heating element 310 can be out of the insufflation gases flow path and does not come into contact with the insufflation gases or the medical instruments inserted through the hollow passage 314. The medical instruments may include a surgical instrument. Isolating the heating element 310 from the insufflation gases flow path by embedding the heating element 310 within the wall of the elongate shaft 304 can reduce and/or avoid contamination, for example, due to the connection of the heating element 310 to wiring that may not be sterile. Isolating the heating element 310 by embedding it within the wall of the elongate shaft 304 can also assist in reducing short circuits.

An electrical wire 316 can be in electrical communication with the heating element 310. The electrical wire 316 can be connected to an end of the heating element 310 closest to the cannula upper housing 302, which can move the wire 316 further away from the patient than a connection location closer to the free end 308 of the elongate shaft 304. The electrical wire 316 can be in electrical communication with the electrical circuitry of a humidifier of an surgical system, for example, any of the surgical systems (e.g., insufflation systems) described above, such that the heating element 310 is powered by a controller of the humidifier. More than one electrical wire can also optionally be connected to the heating element 310. Alternatively, the heating elements 310 may be powered by a further independent controller housed within the humidifier or housed in the insufflator. In a further alternative configuration, the heating element 310 may be powered by any other controller in the surgical system. In the present disclosure, any heating element in the cannula can be controlled by a controller in the insufflator, the cannula, the humidifier, or any other controller external to the insufflator, the cannula, and the humidifier.

The heating element 310 can be configured to transfer heat to the insufflation gases passing through the elongate shaft prior to the insufflation gases exiting the elongate shaft to reach medical instrument inserted through the hollow passage 314 of the elongate shaft 304 of the cannula 300 and/or to a portion of the medical instrument to raise the temperature of the gases and/or the instrument so as to reduce and/or prevent condensation (for example, in the lumen of the elongate shaft 304 and/or on the medical instrument) and/or fogging (for example, on the lens of the medical instrument). The medical instrument may be a surgical instrument. The heating element 310 can increase the temperature of the insufflation gases and/or the medical instrument above a dew point to reduce and/or prevent condensation of the gases and/or reduce (and/or remove) condensation and/or fogging on the medical instrument. The heating element 310 can also allow for better control of the therapy provided by the surgical system.

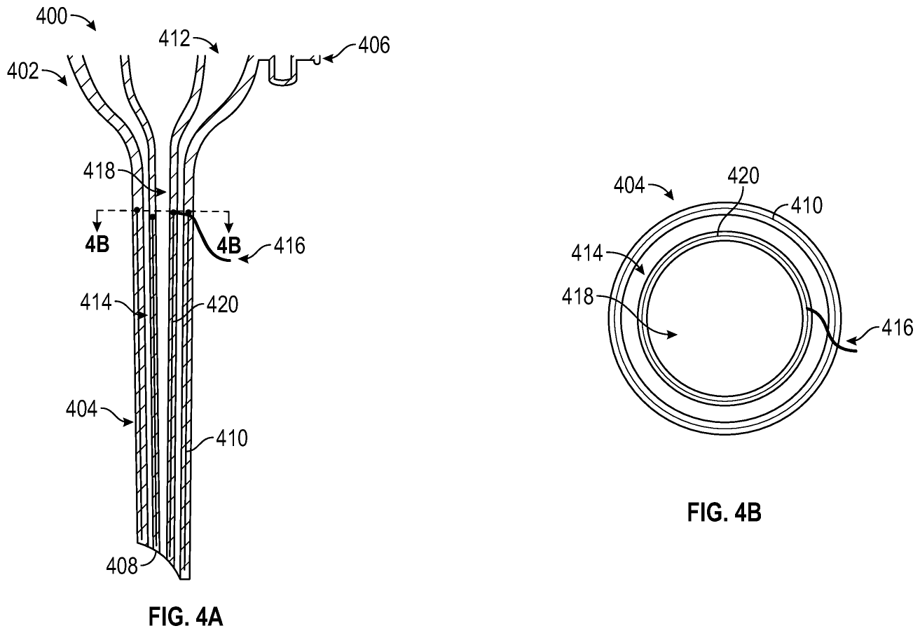

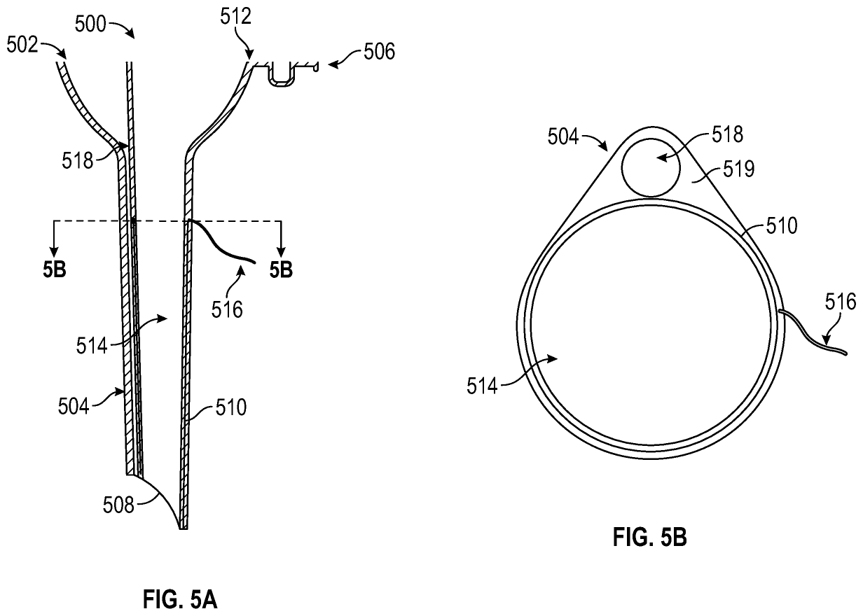

FIGS. 4A-4B and 5A-5B illustrate a surgical cannula 400, 500 that can have any of features of the cannula 300 except that the cannula 400, 500 can include a second hollow passage or lumen 418, 518 extending along the cavity 412, 512 and the hollow passage 414, 514.

As shown in FIGS. 4A and 4B, the second lumen 418 can be located within and generally concentric with the cavity 412 and the hollow passage 414. The inlet 406 may not be in fluid communication with the second lumen 418 so that the insufflation gases may not flow into the second lumen 418. The cannula 400 can include a second heating element 420 located within a wall of the second lumen 418. The second heating element 420 can have any of features of the heating element 310, 410 disclosed herein. For example, the second heating element 420 can extend around or substantially extend around the circumference of the second lumen 418 or extend partially around the circumference of the second lumen 418. The second heating element 420 can be located along a length of the elongate shaft 404 and/or can have substantially the same length as the heating element 410. As the one or more medical instruments are inserted through the second lumen 418, the second heating element 420 can ensure the medical instruments (for example, a medical scope, including but not limited to an optical lens, sensor, or other element on the scope) are heated to prevent, reduce, and/or remove fogging and/or condensation on the medical instrument. The medical instruments may include a surgical instrument. One or more electrical wires 416 can connect to the heating element 410 and the second heating element 420 to energize the heating element 410 and the second heating element 420 as described above.

As shown in FIGS. 5A and 5B, the second lumen 518 can be offset from the cavity 512 and the hollow passage 514. The inlet 516 may not be in fluid communication with the second lumen 518 so that the insufflation gases may not flow into the second lumen 518. As shown in the transverse cross-sectional view in FIG. 5B, the wall of the elongate shaft 504 can have a thicker or inflated section 519 than a remainder of the wall to accommodate the offset second lumen 518. The heating element 510 can extend around or at least substantially around the hollow passage 514 such that the heating element 510 extends through the thicker section 519 of the wall. The close proximity of a portion of the heating element 510 to the offset second lumen 518 can ensure that the medical instruments advanced through the hollow passage 514, and/or the second lumen 518 can be heated to prevent, reduce, and/or remove condensation and/or fogging on the medical instruments. The medical instruments may include a surgical instrument.

The example cannulas shown in FIGS. 4A-4B and 5A-5B can be used for delivering gases to and venting gases from a surgical cavity. The dual lumen cannula can be used to deliver gases and vent smoke/gases simultaneously. The dual lumen cannula can provide a single cannula that can deliver gases and vent gases.

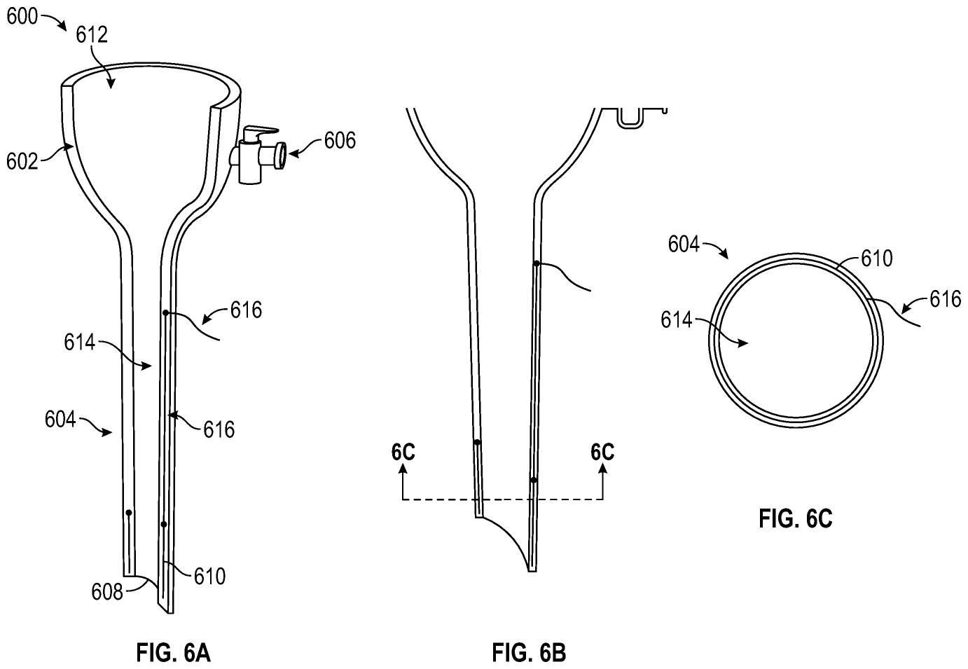

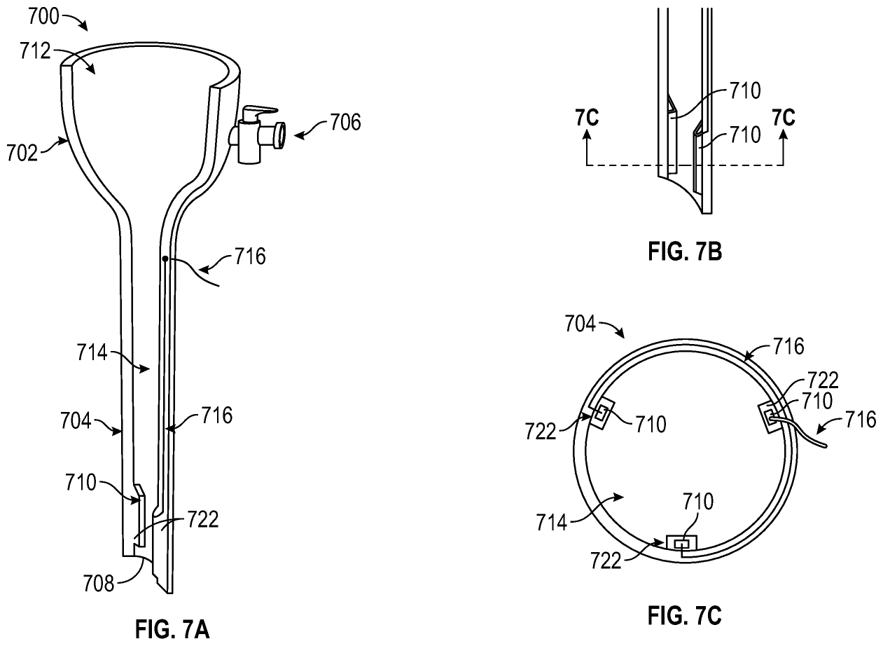

With reference to FIGS. 6A-6C and 7A-7C, the cannula 600, 700 can have any of features of the cannula 300, 400, 500 except as described below. Features of the cannula 600, 700 can be incorporated into features of the cannula 300, 400, 500, and features of the cannula 300, 400, 500 can be incorporated into features of the cannula 600, 700. The heating element 610, 710 can extend along a shorter portion of the elongate shaft 604 than the heating elements shown in FIGS. 3A-5B.

As shown in FIGS. 6A-6C, the heating element 610 can be embedded in the wall of the elongate shaft 604 and can extend from the free end 608 of the shaft 604 for a predetermined length of the elongate shaft 604. The heating element 610 can be disposed adjacent the outlet of the cannula. The heating element may only extend a short distance along the shaft to provide more localized heating (for example, at or near a lens of a scope inserted into the cannula).

As shown in FIGS. 7A-7C, the elongate shaft 704 can include a plurality (for example, two, three, or more) of instrument holders or ribs 722 extending radially inwardly from an inner surface of the hollow passage 714. The ribs 722 can be shorter than the elongate shaft 704. The ribs 722 can be substantially uniformly distributed around the hollow passage 714, or irregularly spaced apart. The instrument holders or ribs 722 can be configured to radially stabilize the medical instrument inserted into the hollow passage 704. The medical instrument may be a surgical instrument. The ribs 722 can be located closer to the free end 708 of the elongate shaft 704 than to a portion of the shaft 704 connected to the cannula upper housing 702. Each rib 722 can include a heating element 710 embedded internally within the rib 722 such that the heating element 710 can be isolated from the gases path. The one or more electrical wires 716 can connect to each of the heating elements 710. Accordingly, the ribs 722 are configured to heat the gases and also are structured to heat the medical instrument inserted within the cannula by conduction. The ribs 722 may either grip the medical instrument or may act as limits to radial movement of the medical instrument within the cannula. The ribs 722 can prevent the medical instrument from resting against the walls of the cannula. The ribs 722 can maintain the medical instrument in a substantially concentric arrangement relative to the hollow passage 714. The ribs 722 can ensure that heated gases flow around the medical instrument when the medical instrument is inserted in the hollow passage 714.

The ribs may also be elongate and may extend the length of the cannula shaft. The heating element in the ribs can transmit heat to the cannula by conduction (for example, by contacting the gases and/or the medical instrument). The ribs may also optionally be near the outlet of the cannula, near the entrance to the cannula shaft, or at some other region along the cannula shaft.

The cannula may also include multiple sets of the ribs. Each set of ribs may include a plurality of ribs. One or more ribs in each of the sets may include a heating element disposed within the ribs. For example, each alternating rib in each set may include a heating element. A first set of ribs can be located at an upper region closer to (for example, adjacent) the entrance to the cannula shaft. A second set of ribs can be disposed closer to (for example, adjacent) the outlet of the cannula. The multiple sets of ribs can be spaced apart from each other.

The one or more electrical wires 616, 716 connecting to the heating element(s) 610, 710 can extend along and/or through (for example, being overmoulded in) the wall of the elongate shaft 604, 704. The electrical wires 616, 716 can extend from the heating element(s) 610, 710 toward the cannula upper housing 602, 702. The one or more electrical wires 616, 716 can exit the wall of the elongate shaft 604, 704 at or near a base of the cannula upper housing 602, 702 and can be in electrical communication with electrical circuitry of a humidifier to energize the heating element(s) 610, 710. The heating element can be controlled by a controller in the insufflator, the cannula, the humidifier, or any other controller external to the insufflator, the cannula, and the humidifier. The exit location of the one or more electrical wires 616, 716 on the elongate shaft 604, 704 can ensure that the wires 616, 716 extend from the elongate shaft 604, 704 at a location further away from the patient than the location of the heating element(s) 610, 704 to reduce the likelihood of the wires 616, 716 contacting the patient and/or an outer surface of the elongate shaft 604, 704.

The location and/or length of the heating element 610, 710 can allow the heating element 610, 710 to be closer to where the lens of a medical scope would be located. The heating element 610, 710 can thus heat more localized areas of the cannula 600, 700 than the heating elements 310, 410, 510 described above to target the fogging and/or condensation more directly and/or effectively, and/or with reduced potential harm to the patient (for example, by providing reduced power to the heating element(s) so the portion of the elongate shaft within the surgical cavity can be not too hot).

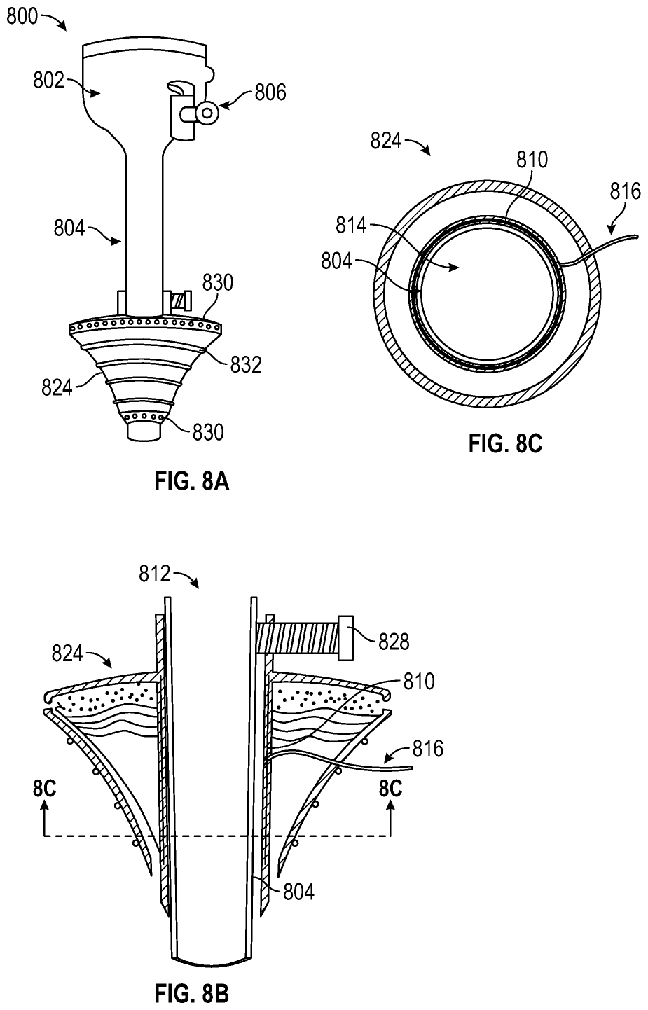

FIGS. 8A-8C illustrate another example cannula 800 with a heating element 810 configured to provided more localized heating along a partial length of the elongate shaft 804. The cannula 800 can have any of features of the cannula 300, 400, 500, 600, 700 described above. Features of the cannula 800 can be incorporated into features of the cannula 300, 400, 500, 600, 700 and features of the cannula 300, 400, 500, 600, 700 can be incorporated into features of the cannula 800.

The heating element 810 can be located within a sleeve attachment 824 configured to be coupled to the elongate shaft 804. The sleeve attachment 824 can include one or more vents 830 to provide venting of the gases and/or surgical smoke. Insufflation gases can enter the surgical cavity via the cannula. The vents 830 (for example, located near or at both the proximal and distal ends of the sleeve attachment 824) can define a fluid path for smoke and/or other gases to exit from the surgical cavity. The sleeve attachment 824 may include one or more filter elements within the attachment 824 (for example, closer to the vents 830 near or at the proximal end of the attachment 824). The filter elements can filter out undesirable smoke, gases, and/or odor, for example. The heating element in the sleeve attachment 824 can heat the filter elements in order to reduce and/or prevent condensation and/or clogging in the filter elements. The sleeve attachment 824 can have features that help to position and/or retain the cannula 800 inside the surgical cavity. For example, the sleeve attachment 824 can have an outer shape of generally a funnel, and/or can include a plurality of ridges 832 on an outer surface of the sleeve attachment. The ridges 832 can assist in retaining the cannula 800 and/or the sleeve attachment 824 within a surgical cavity. The sleeve attachment 824 can include a lumen configured to slidably receive the elongate shaft 804 so that the sleeve attachment 824 circumferentially surrounds a portion of the elongate shaft 804. The sleeve attachment 824 can be securely attached to the elongate shaft 804 by a set screw 828, which can provide a radial pressure against the outer surface of the elongate shaft 804 when the screw 828 is tightened onto the elongate shaft 804. Other securement features can also be used to secure the sleeve attachment 824 to the shaft 804.

As shown in FIGS. 8B and 8C, the heating element 810 can be located adjacent an inner wall of the sleeve attachment 824 and can be in electrical communication with one or more electrical wires 816 extending out of the outer surface of the sleeve attachment 824. This location of the heating element 810 can allow the heating element 810 to be as closer to the elongate shaft 804 while still being out of the gases flow path. The heating element 810 can transfer heat to the gases passing through the hollow passage 814 and/or over the medical instrument to prevent, reduce, and/or remove fogging and/or condensation on the lens. The medical instrument may be a surgical instrument. The sleeve attachment 824 can be attached to a location on the elongate shaft 804 such that the heating element 810 is closer to the lens of the medical instrument to provide more efficient prevention, reduction, and/or removal of the fogging and/or condensation. The sleeve attachment 824 can also optionally be attached to other locations of the elongate shaft 804 to provide more localized heating at those locations.

### Examples of a Heater Associated with a Cannula Upper Housing

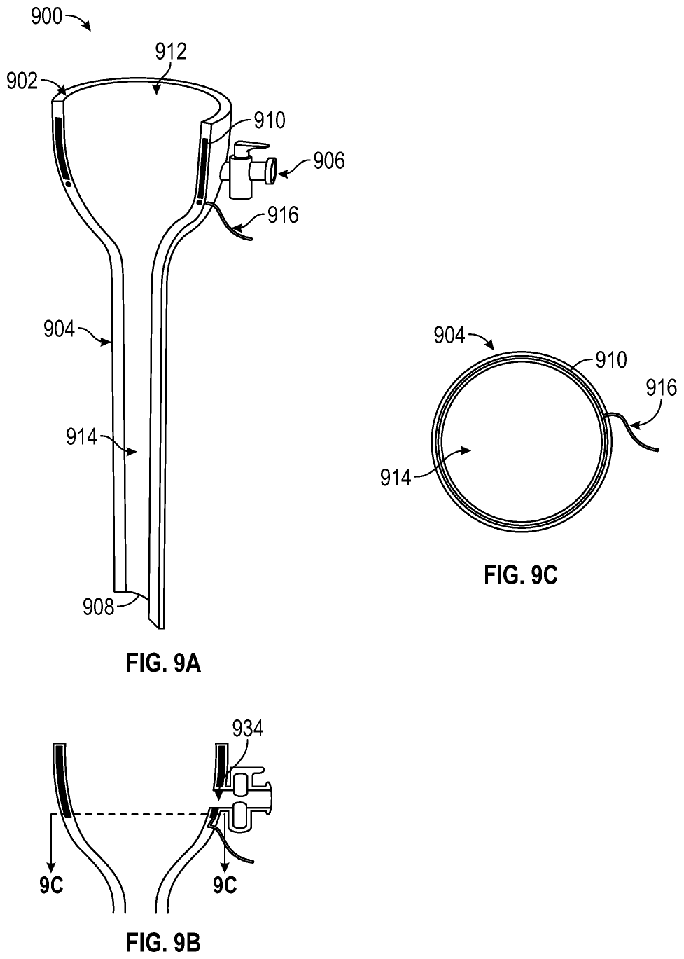

FIGS. 9A-9C illustrate an example cannula 900 with the heating element 910 located along the cannula upper housing 902. FIG. 9A shows a perspective view of the cannula 900 sectioned along a central longitudinal plane to better illustrate the heating element 910. The cannula 900 can have any of features of the cannula 300, 400, 500, 600, 700, 800 described above. Features of the cannula 900 can be incorporated into features of the cannula 300, 400, 500, 600, 700, 800 and features of the cannula 300, 400, 500 600, 700, 800 can be incorporated into features of the cannula 900.

As shown in FIGS. 9A-9C, the cannula 900 can include a cannula upper housing 902 and an elongate body 904 extending from the body 902. The cannula upper housing 902 can house one or more instrument seals. A free end 908 of the elongate body 904 can optionally have a pointed or otherwise sharp end 908 or a square tip. The cannula upper housing 902 can have an opening 912, which can be in fluid communication with (for example, being connected to or continuous with) a hollow passage 914 of the elongate shaft. The cannula 900 can include a gases inlet 906 coupled to a wall of the cannula upper housing 902. The gases inlet 906 can be connected to a gases delivery tube of an surgical system (for example, insufflation systems, or any of the systems disclosed herein). As shown in FIG. 9B, the inlet 906 can be in fluid communication with the opening 912 of the cannula upper housing 902 and/or the hollow passage 914 of the elongate shaft 904.

The heating element 910 can be embedded in a wall of the cannula upper housing 902. The heating element 910 and/or other heating elements in this disclosure can be attached and/or disposed on an inner wall of the cannula (for example, the inner wall of the upper housing and/or the inner wall of the shaft). Alternatively, the heating element examples may be wrapped around the outer surface of the cannula shaft. The heating element 910 can be moulded into the wall of the cannula upper housing 902. As shown in FIGS. 9A and 9B, the heating element 910 can extend along at least a length of the cannula upper housing 902. The heating element 910 can be flexible. As shown in FIG. 9C, the heating element 910 can be commensurate with a cross-sectional profile of the cannula upper housing 902. The heating element 910 can extend circumferentially or at least substantially circumferentially around the opening 912 of the cannula upper housing 902. The heating element 910 can be out of the insufflation gases flow path and does not come into contact with the insufflation gases or the medical instruments inserted through the opening 912. The medical instruments may include a surgical instrument. The heating element 910 can also include a gap 934 to allow the inlet 906 to extend through the wall of the cannula upper housing 902. Isolating the heating element 910 from the insufflation flow path by embedding the heating element within the wall of the elongate shaft can reduce and/or avoid contamination.

An electrical wire 916 can be in electrical communication with the heating element 910. The electrical wire 916 can be connected to an end of the heating element 910 (for example, the end closer to the elongate shaft 910 as shown in FIG. 9B). The electrical wire 916 can be further away from the patient to reduce the likelihood of the wire 916 contacting the patient than a wire connecting to a shaft heating element, for example, the shaft heating elements described above. The electrical wire 916 can be in electrical communication with the electrical circuitry of a humidifier of an surgical system, for example, insufflation systems, or any of the surgical systems described above, such that the heating element 910 is powered by a controller of the humidifier. More than one electrical wire can also optionally be connected to the heating element 910. The heating element can be controlled by a controller in the insufflator, the cannula, the humidifier, or any other controller external to the insufflator, the cannula, and the humidifier.

The heating element 910 can be configured to transfer heat to the insufflation gases passing from the inlet 906 into the opening 912 and/or to a portion of the medical instrument inserted into the opening 912 (for example, when the instrument is advanced into and/or removed from the cannula 900) to raise the temperature of the instrument so as to reduce and/or prevent condensation and/or fogging (for example, on the lens). The heating element 910 can increase the temperature of the insufflation gases and/or the medical instrument above a dew point to prevent condensation of the gases and/or reduce (and/or remove) condensation and/or fogging on the medical instrument. The heating element 910 can also allow for better control of the therapy provided by the surgical system. Locating the heating element 910 within the cannula upper housing 902 can allow the cannula 900 to be heated in a manner that is safer for the patient as the cannula 900 is not as hot at or near the free end 908 (or the patient interface end), and/or can be heated to a higher temperature to prevent, reduce, and/or remove fogging and/or condensation.

In some configurations, a filter or filter elements can be connected to the cannula upper housing 902, for example, prior to the gases inlet 906. Alternatively, filters may be integrated into the cannula (for example, to the cannula upper housing 902) and can be located in the gases flow path. One or more heater elements, for example, the heating element 910, can be positioned in contact with or integrated into the filter or filter elements to heat the filter or filter elements and prevent the filter or filter elements from clogging. The heating of the filter or filter elements to prevent condensation and/or clogging can extend the life of the filter or filter elements and maintain efficiency of the filter or filter elements.

### Additional Examples of a Cannula Heater

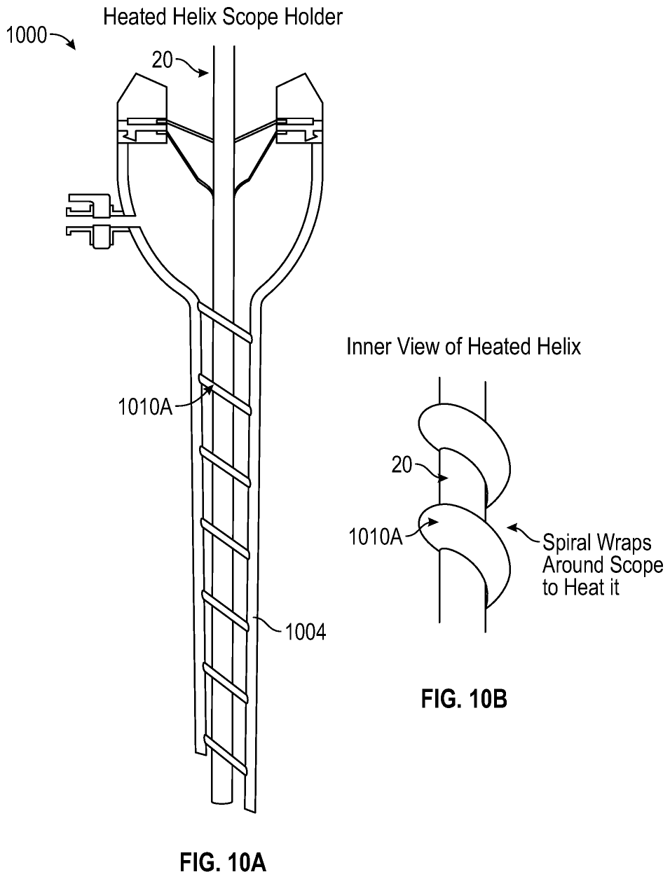

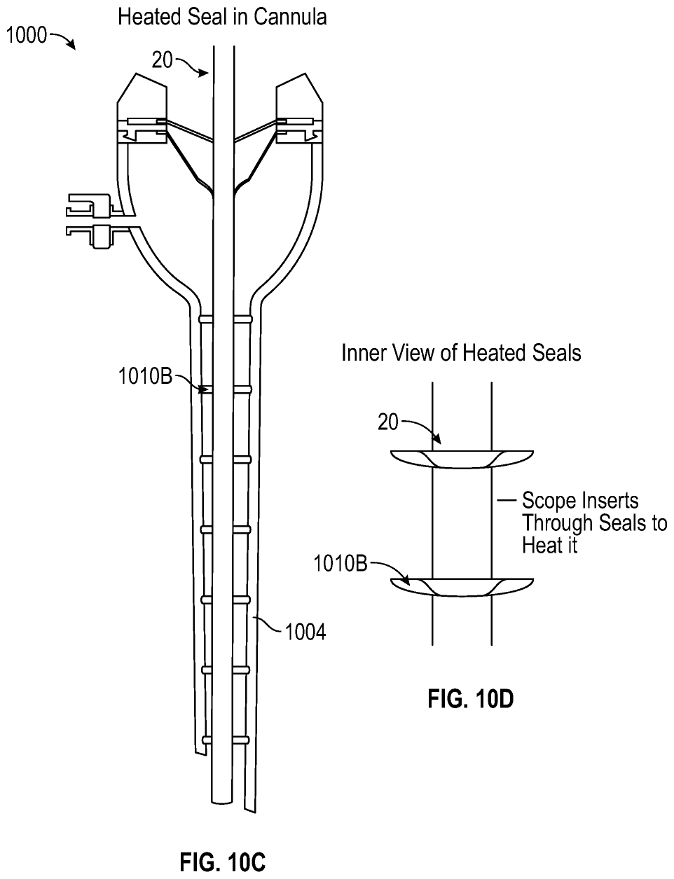

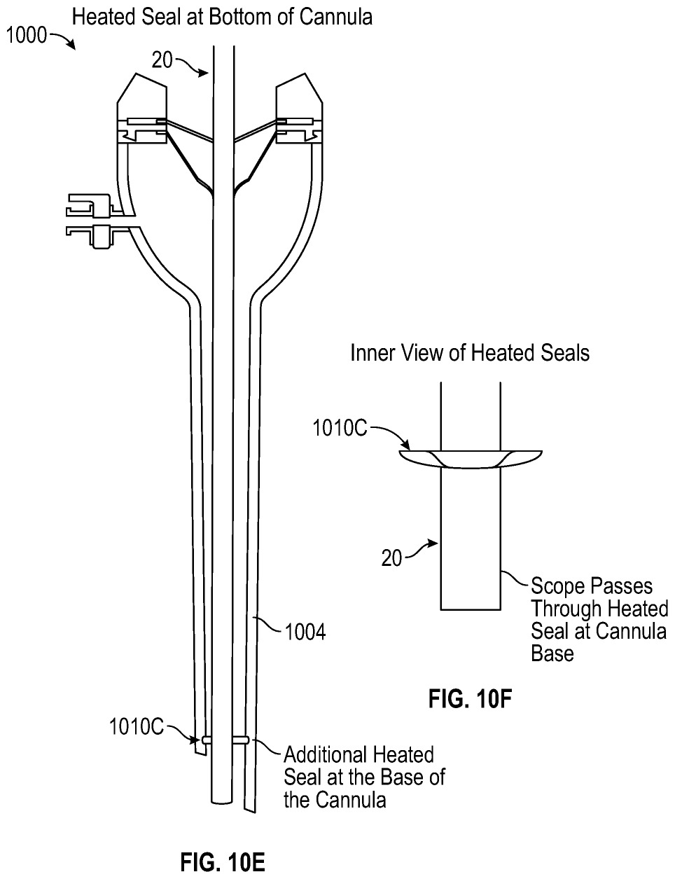

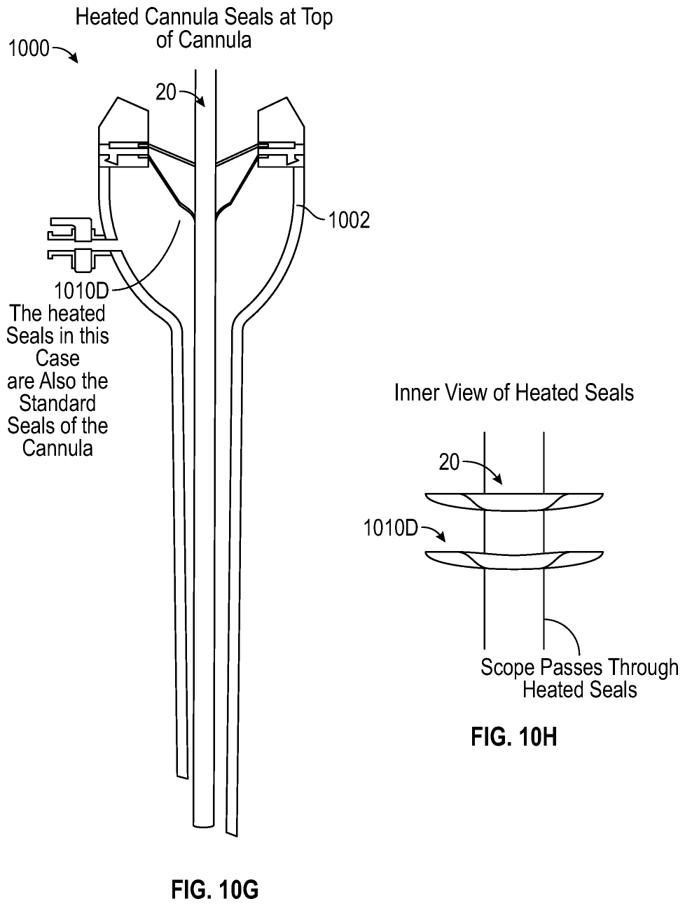

As shown in FIGS. 10A-10H, one or more internal flaps can be included in the cannula 1000. The cannula 1000 can have any of the features of the cannula 300, 400, 500, 600, 700, 800, 900 described above and the cannula 300, 400, 500 600, 700, 800, 900 can have any of the features of the cannula 1000. The one or more of the seals can include a heating element. The seals can be in contact with a medical instrument inserted into the cannula. The medical instrument may be a surgical instrument. The heating elements in the seals can heat the instrument to reduce and/or remove fogging on the instrument. The heating elements can also heat the gases passing through the seals.

The one or more internal flaps can be integrated in the cannula 1000 (for example, overmoulded to an inner wall of the elongate shaft 1004) or can be removably inserted into the cannula 1000. As shown in FIGS. 10A and 10B, the internal flaps can form a heated helical instrument holder 1010A. The helical instrument holder 1010A can extend radially inward from an inner wall of the elongate shaft 1004 of the cannula 1000. The helical instrument holder 1010A can extend along substantially an entire length of the elongate shaft 1004, for example, shown in FIG. 10A, or extend along a portion or portions of the elongate shaft 1004. When the instrument 20 is inserted into the cannula 1000, the helical instrument holder 1010A can spirally wrap around the instrument 20 to heat the instrument 20. The internal flaps can also be attached to a scope, a cannula, or the like.

As shown in FIGS. 10C and 10D, the internal flaps can include a plurality of heated vanes 1010B that can be distributed along substantially an entire length of the elongate shaft 1004. The heated vanes 1010B can also be distributed along a portion or portions of the elongate shaft 1004. As shown in FIGS. 10E and 10F, a single heated vane 1010C can be located at or near the outlet of the cannula 1000. When the instrument 20 is inserted into the cannula 1000, the heated vane(s) 1010B, 1010C can seal around the instrument 20 to heat the instrument 20.

As shown in FIGS. 10G and 10H, the standard cannula seals 1010D located at the opening of the cannula upper housing 1002 can serve as the internal flaps. When the instrument 20 is inserted into the cannula 1000, the standard cannula seals 1010D can seal around the instrument 20 to heat the instrument 20.

In some configurations, the cannula can incorporate two or more of the internal flaps disclosed herein. For example, the standard cannula seals 1010D can be used in combination with the helical instrument holder 1010A or the heated vane(s) 1010B, 1010C. As another example, the helical instrument holder 1010A can be used in combination with the additional heater vane 1010C.

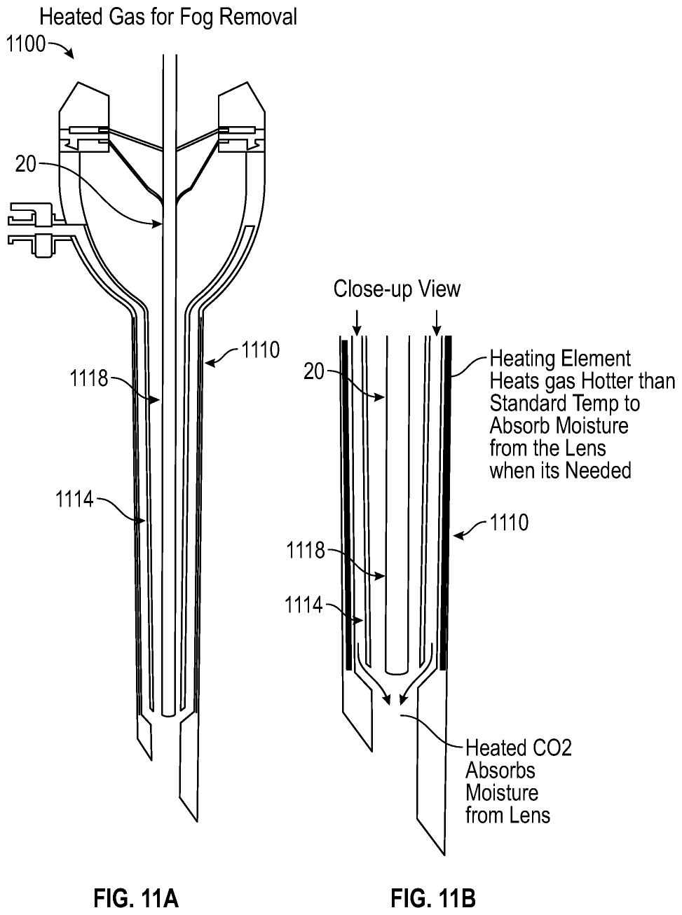

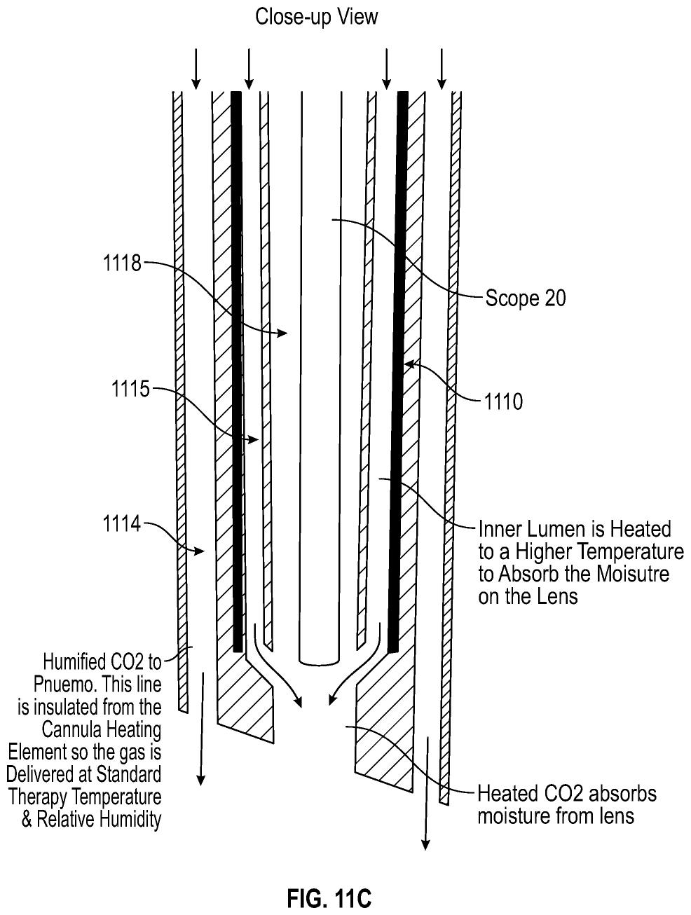

FIGS. 11A-11C illustrate heating of gases flowing past the cannula 1100 having concentric multi-lumens. The arrows indicate the direction of the gases flow. The cannula 1100 can have an inner lumen 1118 and an outer lumen 1114. As shown in FIGS. 11A and 11B, the heating element 1110 can be located (for example, molded) in the wall (for example, the outer wall) of the outer lumen 1114. Gases (for example, humidified insufflation gases) can be introduced into the outer lumen 1114 of the cannula 1100. The medial instrument 20 can be inserted into the inner lumen 1118. The heating element 1110 can be configured to heat the gases to a temperature higher than the standard therapy gases temperature and/or relative humidity, which may be, for example, of dry cold gas, dry hot gas, humidified cold gas, or humidified hot gas. When the heated gases meet the instrument 20, for example, near the outlet of the cannula 1100, the heated gases can absorb the moisture on the instrument 20.

As shown in FIG. 11C, the cannula 1100 can have an inner lumen 1118, an intermediate lumen 1115, and an outer lumen 1114. The heating element 1110 can be located (for example, molded) in the wall (for example, the outer wall) of the intermediate lumen 1115. Gases (for example, humidified insufflation gases) can be introduced into the outer lumen 1114 and the intermediate lumen 1115 of the cannula 1100. The heating element 1110 can be configured to heat the gases in the intermediate lumen 1115 to a temperature higher than the standard therapy gases temperature. When the heated gases in the intermediate lumen 1115 meet the instrument 20, for example, near the outlet of the cannula 1100, the heated gases can absorb the moisture on the instrument 20. The gases in the outer lumen 1114 can be insulated from the heating element 1110 so that the gases in the outer lumen 1114 can be delivered at a standard therapy temperature and/or relative humidity.

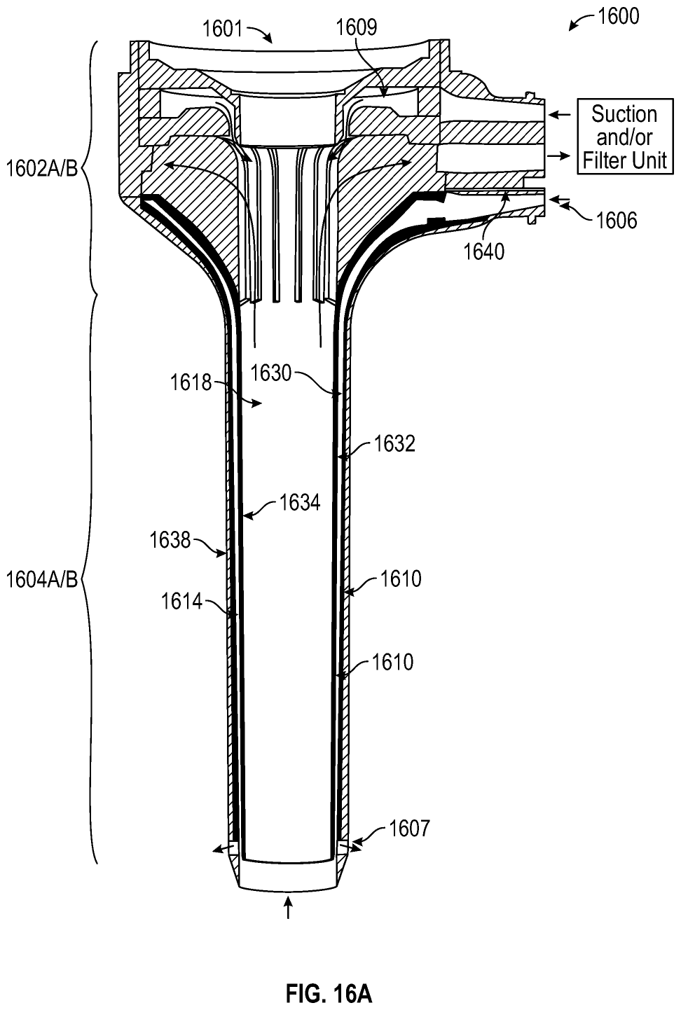

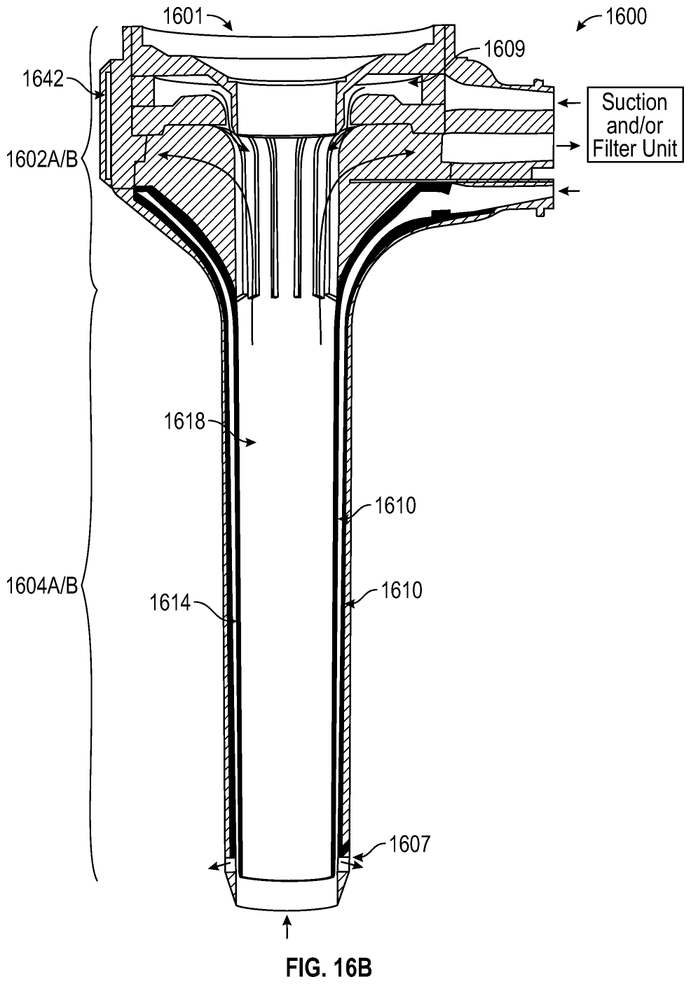

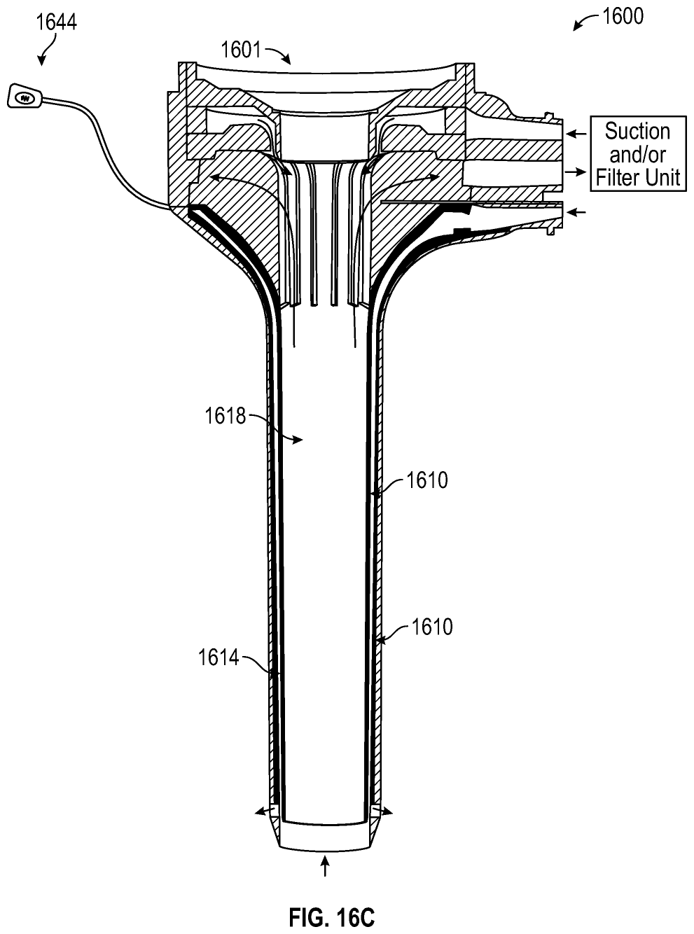

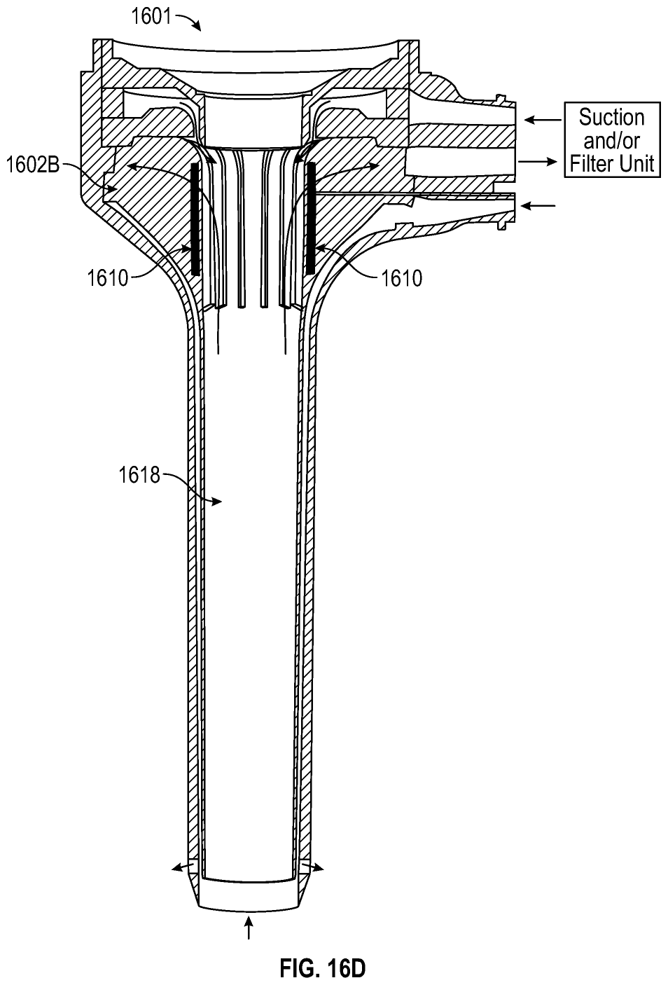

FIGS. 16A-16D illustrate heating of gases flowing past a cannula 1600 having generally concentric multi-lumens. The arrows indicate the direction of the gases flow, including the flow of an insufflation gas and the flow of gases from the surgical cavity. The cannula 1600 can include an inner tubular member and an outer tubular member. The outer tubular member can include an outer body 1602A and an outer elongate shaft 1604A extending distally from the outer body 1602A. The inner tubular member can include an inner body 1602B and an inner elongate shaft 1604B extending distally from the inner body 1602B. A lumen of the inner tubular member can define an inner lumen 1618. A medical instrument (for example, a scope, or any other instruments disclosed herein) can be inserted into the inner lumen 1618. The medical instrument may be a surgical instrument.

An outer surface 1632 of the inner tubular member and an inner surface 1630 of the outer tubular member can define an outer lumen 1614 of the cannula 1600. The inner lumen 1618 and the outer lumen 1614 can be substantially coaxial (such as, for example coaxial) in some configurations. The inner lumen 1618 and the outer lumen 1614 can be substantially concentric (such as, for example, concentric) in some configurations.

Fluids including liquids or gases, for example, humidified insufflation gases, can be introduced can be introduced into the outer lumen 1614 of the cannula 1600. The outer lumen 1614 can define an insufflation passage, which has an inlet for the insufflation gases via the insufflation port 1606. As shown in FIGS. 16A-16D, the outer lumen 1614 can terminate at its distal end at an opening 1607. The opening 1607 can be the sole outlet of the insufflation gases from the insufflation passage. The opening 1607 can also optionally include a plurality of apertures. The opening or plurality of apertures can be located on a wall of the outer tubular member, for example, at least at a distal portion of the cannula 1600, or anywhere along the outer elongate shaft 1604A that is configured to be inserted within the surgical cavity.

The cannula 1600 can be sealed pneumatically so that ambient air does not enter the surgical cavity, for example, via the inner lumen 1618. The inner lumen 1618 can allow gases inside the surgical cavity to move proximally toward the inner body 1602B. The cannula 1600 can be coupled to a suction and/or filtering unit so that the gases from the surgical cavity can move proximally through the inner lumen 1618. At least a portion of the gases from the surgical cavity be returned to the inner body 1602B via a recirculation loop to an inlet 1609 leading from the suction and/or filtering unit back to the inner body 1602B. The recirculated gases can create a region, similar to an air curtain or barrier, which substantially prevents entrainment of room or ambient air into the inner lumen 1618.

A heat exchanging process can occur between the incoming insufflation gases and the recirculated gases. A net heat transfer may exist between the outer lumen 1614 and the inner lumen 1618. Condensation can form if the temperature of the insufflation gases falls below the dew point. For example, insufflation gases that are humidified above a temperature of the gases in the surgical cavity coming in through the outer lumen 1614 may be cooled down by the colder recirculated gases. This cooling may cause condensation to form in the outer lumen 1614. The condensation can reduce the therapeutic effect of the incoming insufflation gases.

A heating element may be located in the cannula 1600. Heating element(s) can be used to advantageously maintain or elevate the incoming insufflation gases above the dew point. The heating elements may be located in the upper housing and/or cannula shaft. The heating element may be located in the inner tubular member and/or the outer tubular member. As shown in FIGS. 16A-16C, the heating element 1610 can be located (for example, molded) anywhere between the outer surface 1638 of the outer tubular lumen, including at least portions of the outer body 1602A and/or the outer elongate shaft 1604A, and/or the inner surface 1634 of the inner tubular lumen, including at least portions of the inner body 1602B and/or the inner elongate shaft 1604B. The heating element can be embedded, partially or entirely, within the wall of the outer tubular lumen, including at least portions of the outer body 1602A and/or the outer elongate shaft 1604A, and/or within the wall of the inner tubular lumen, including at least portions of the inner body 1602B and/or the inner elongate shaft 1604B. The heating element can also be located within the insufflation passage instead of being embedded in the wall. The heating element can run generally parallel to the insufflation passage. The heating element can thus be integrated in a wall that contacts, or is in very close proximity to, the incoming insufflation gas.

Power can be supplied to the heating element 1610 via the gases delivery conduit disclosed herein, for example, with a lead wire 1640 extending from the heating element 1610 to the insufflation port 1606 (FIG. 16A), via an integrated power supply, for example, battery 1642 in or on the cannula (FIG. 16B), or via an electrical connector or power plug 1644 (FIG. 16C).

Optionally, as shown in FIG. 16D, the heating element 1610 can be located away and isolated from the incoming insufflation gas. As shown in FIG. 16D, the heating element 1610 can be located at or near an inner surface of the inner body 1602B. The heating element 1610 can heat the medical instrument that is inserted via the inner lumen 1618 to the surgical cavity. The medical instrument can be a surgical instrument, such as an imaging unit (for example, the scope, camera, and the like), or otherwise.

### Examples of Heating Elements

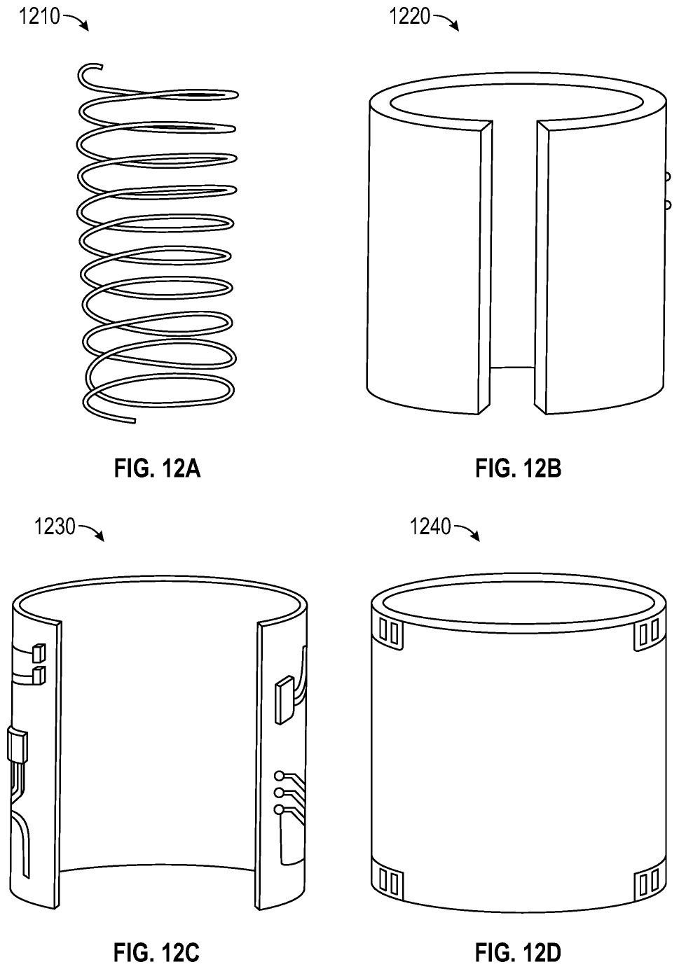

Examples of heating elements will be described with reference to FIGS. 12A-12D. These examples of heating elements can be implemented as the sole or first heating element and/or second heating element, or more than two heating elements, in any of the heated cannula examples disclosed herein. The heating element can have an arcuate shape.

As shown in FIG. 12A, the heating element can include a heater wire 1210. The heater wire 1210 can be helical and/or can helically extend around the hollow passage of the elongate shaft, the second lumen, and/or the cavity of the cannula upper housing. As shown in FIG. 12B, the heating element can include a flexible band heater 1220. As shown in FIG. 12C, the heating element can include a flexible printed circuit board (“PCB”) 1230 or a rigid PCB pre-shaped to an arcuate shape. As shown in FIG. 12D, the heating element can include a thermo-elastic and/or thermal-electric plastic material (e.g., a conductive plastic). The thermo-elastic and thermal-electric plastic material can form a planar sheet 1240 that is bendable and/or malleable, and/or can generate or dissipate heat when a current is applied to the material. The dimensions of the example heating elements can be varied to accommodate the different locations in the cannula (and/or sleeve attachment) disclosed herein.

### Additional Power Supply Examples

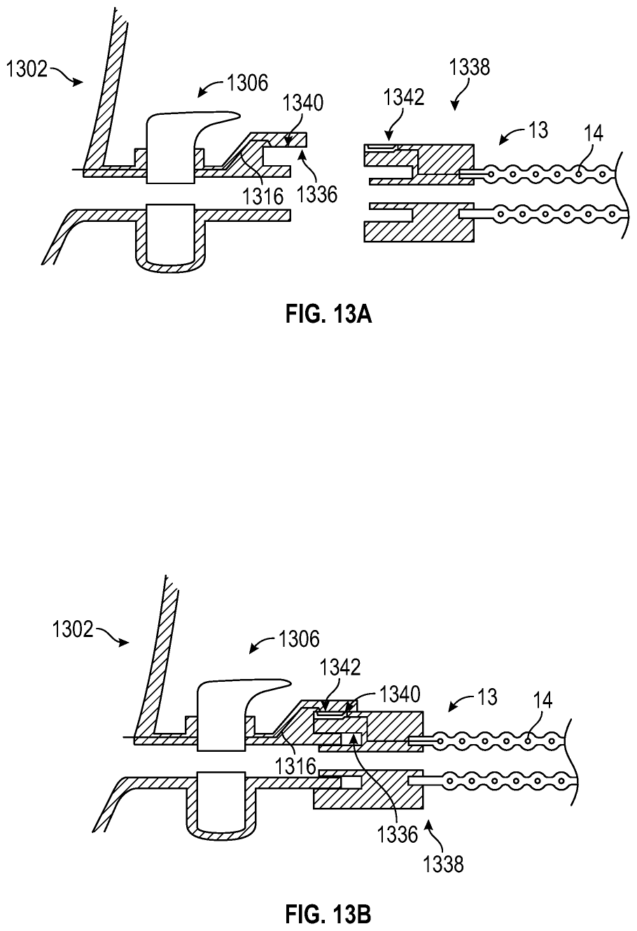

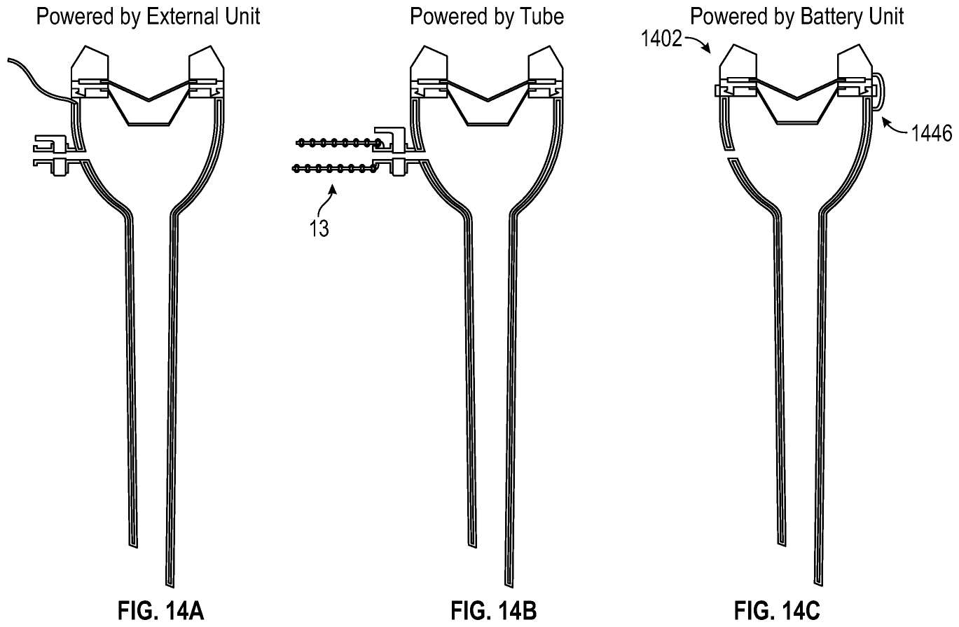

The example cannulas disclosed herein can also include a socket connection for supplying power to the heating element via the one or more electrical wires. As shown in FIGS. 13A-13B, the gases inlet 1306 of the cannula can include an electrical connector 1336. The electrical connector 1336 can be in electrical communication with the one or more electrical wires 1316. The one or more electrical wires 1316 can be embedded within (for example, overmoulded in) the wall of a partial length of the cannula upper housing 1302 and also optionally the wall of a partial length of the elongate shaft, extending between the heating element in the cannula and the electrical connector 1336.

The electrical connector 1336 can be configured to couple to a corresponding connector 1338 (for example, a socket connector) on a gases delivery tube 13 of a surgical system (for example, an insufflator system, or any other surgical systems disclosed herein) to supply power to the heating element. The gases delivery tube 13 can include a helically wound tube that is moulded into the corresponding connector 1338, which can include a hard plastic material. Alternatively, the gases delivery tube 13 can include a non-helical or straight tube. Optionally, the gases delivery tube 13 can be corrugated or non-corrugated. The socket connection can secure the gases delivery tube 13 to the cannula. As shown in FIGS. 13A and 13B, the electrical connector 1336 can include a pin 1340 configured to be coupled to a PCB edge connector 1342 of the corresponding connector 1338 to establish electrical communication between the heating element and the heater wire circuit 14 of the gases delivery tube 13.

As shown in FIGS. 14A-14C, the heating elements disclosed herein can be powered by one or more power source options, for example, by an external power unit (FIG. 14A), by being in electrical connection with a heater wire in the gases delivery tube 13 (FIG. 14B), and/or by a battery unit 1446 mounted on the cannula (for example, on the upper housing 1402 as shown in FIG. 14C). Optionally, the heating element can include an inductive heating element. Optionally, the heating element can include a chemical heating element, for example, including but not limited to silica beads. Optionally, the cannula can be pre-heated prior to insertion.

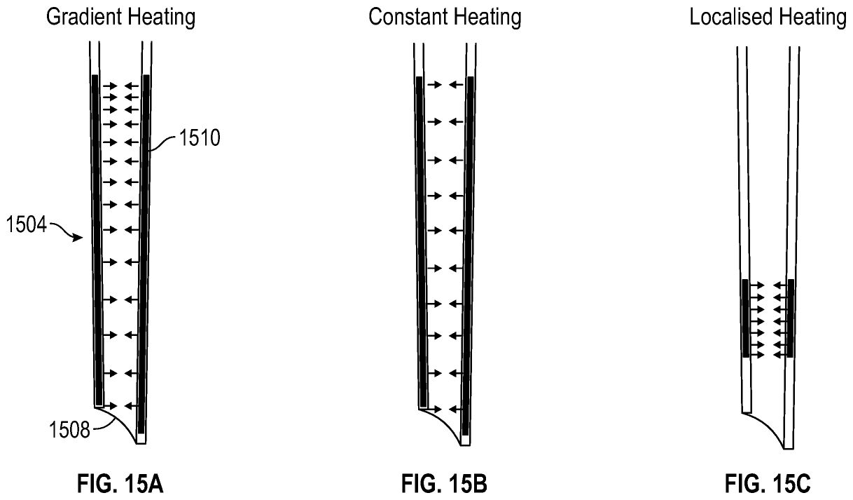

As shown in FIGS. 15A-15C, the heating effect of the heating elements disclosed herein can be varied. The heating elements can provide gradient heating. For example, as shown in a heating element 1510 located in a portion of the cannula shaft 1504, the amount of heat transferred can decrease toward the outlet of the cannula 1508. The amount of heat transferred can also increase toward the outlet of the cannula 1508. The heating elements can provide substantially constant or uniform heating along a longitudinal axis of the cannula (FIG. 15B). The heating element can also provide localized heating as shown in FIG. 15C (for example, by a localized heating element disclosed herein). The heating elements examples shown in FIGS. 15A-15C can also be incorporated into the internal flaps in FIGS. 10C and 10D.

Heating can be varied across the cannula shaft. Alternatively, heating can be temporally varied, that is, over time. For example, the cannula can be rapidly heated up to a set point and heating can then be controlled to maintain the set point. Alternatively, the heating element in the cannula can undergo a warm-up function that slowly ramps up the temperature. Alternatively, heating can be ramped over a specific warm-up period.

The heating elements disclosed herein can be controlled by the controller in the humidifier and can communicate with the controller in the humidifier. Alternatively, heating elements disclosed herein can be controlled by a separate independent control unit. The heating elements can also optionally be in communication with the insufflator and may be controlled by the controller in the insufflator.

### Terminology

Examples of medical gases delivery systems and associated components and methods have been described with reference to the figures. The figures show various systems and modules and connections between them. The various modules and systems can be combined in various configurations and connections between the various modules and systems can represent physical or logical links. The representations in the figures have been presented to clearly illustrate the principles and details regarding divisions of modules or systems have been provided for ease of description rather than attempting to delineate separate physical embodiments. The examples and figures are intended to illustrate and not to limit the scope of the inventions described herein. For example, the principles herein may be applied to a surgical humidifier as well as other types of humidification systems, including respiratory humidifiers. However, the humidification systems and methods may also optionally not involve a patient's respiratory system and may not be placed within a portion of the respiratory tract (for example, nose, mouth, trachea, and/or bronchi).

As used herein, the term “processor” refers broadly to any suitable device, logical block, module, circuit, or combination of elements for executing instructions. For example, the controller 8 can include any conventional general purpose single- or multi-chip microprocessor such as a Pentium® processor, a MIPS® processor, a Power PC® processor, AMD® processor, ARM® processor, or an ALPHA® processor. In addition, the controller 122 can include any conventional special purpose microprocessor such as a digital signal processor or a microcontroller. The various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein, or can be a pure software in the main processor. For example, logic module can be a software-implemented function block which does not utilize any additional and/or specialized hardware elements. Controller can be implemented as a combination of computing devices, e.g., a combination of a DSP and a microprocessor, a combination of a microcontroller and a microprocessor, a plurality of microprocessors, one or more microprocessors in conjunction with a DSP core, or any other such configuration.

Data storage can refer to electronic circuitry that allows data to be stored and retrieved by a processor. Data storage can refer to external devices or systems, for example, disk drives or solid state drives. Data storage can also refer to fast semiconductor storage (chips), for example, Random Access Memory (RAM) or various forms of Read Only Memory (ROM), which are directly connected to the communication bus or the controller. Other types of data storage include bubble memory and core memory. Data storage can be physical hardware configured to store data in a non-transitory medium.

Although certain embodiments and examples are disclosed herein, inventive subject matter extends beyond the specifically disclosed embodiments to other alternative embodiments and/or uses, and to modifications and equivalents thereof. Thus, the scope of the claims or embodiments appended hereto is not limited by any of the particular embodiments described herein. For example, in any method or process disclosed herein, the acts or operations of the method or process can be performed in any suitable sequence and are not necessarily limited to any particular disclosed sequence. Various operations can be described as multiple discrete operations in turn, in a manner that can be helpful in understanding certain embodiments; however, the order of description should not be construed to imply that these operations are order dependent. Additionally, the structures described herein can be embodied as integrated components or as separate components. For purposes of comparing various embodiments, certain aspects and advantages of these embodiments are described. Not necessarily all such aspects or advantages are achieved by any particular embodiment. Thus, for example, various embodiments can be carried out in a manner that achieves or optimizes one advantage or group of advantages as taught herein without necessarily achieving other aspects or advantages as can also be taught or suggested herein.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments. As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is otherwise understood with the context as used in general to convey that an item, term, etc. may be either X, Y or Z. Thus, such conjunctive language is not generally intended to imply that certain embodiments require at least one of X, at least one of Y and at least one of Z each to be present. As used herein, the words “about” or “approximately” can mean a value is within ±10%, within ±5%, or within ±1% of the stated value.

Methods and processes described herein may be embodied in, and partially or fully automated via, software code modules executed by one or more general and/or special purpose computers. The word “module” refers to logic embodied in hardware and/or firmware, or to a collection of software instructions, possibly having entry and exit points, written in a programming language, such as, for example, C or C++. A software module may be compiled and linked into an executable program, installed in a dynamically linked library, or may be written in an interpreted programming language such as, for example, BASIC, Perl, or Python. It will be appreciated that software modules may be callable from other modules or from themselves, and/or may be invoked in response to detected events or interrupts. Software instructions may be embedded in firmware, such as an erasable programmable read-only memory (EPROM). It will be further appreciated that hardware modules may comprise connected logic units, such as gates and flip-flops, and/or may comprised programmable units, such as programmable gate arrays, application specific integrated circuits, and/or processors. The modules described herein can be implemented as software modules, but also may be represented in hardware and/or firmware. Moreover, although in some embodiments a module may be separately compiled, in other embodiments a module may represent a subset of instructions of a separately compiled program, and may not have an interface available to other logical program units.

In certain embodiments, code modules may be implemented and/or stored in any type of computer-readable medium or other computer storage device. In some systems, data (and/or metadata) input to the system, data generated by the system, and/or data used by the system can be stored in any type of computer data repository, such as a relational database and/or flat file system. Any of the systems, methods, and processes described herein may include an interface configured to permit interaction with users, operators, other systems, components, programs, and so forth.

It should be emphasized that many variations and modifications may be made to the embodiments described herein, the elements of which are to be understood as being among other acceptable examples. All such modifications and variations are intended to be included herein within the scope of this disclosure and protected by the following claims. Further, nothing in the foregoing disclosure is intended to imply that any particular component, characteristic or process step is necessary or essential.

1. A surgical cannula for providing insufflation gases to a surgical cavity and providing a passage for insertion of one or more medical instruments, the cannula comprising:
a cannula upper housing including an opening;
an elongate shaft extending from the cannula upper housing, the shaft defining a hollow passage to provide the insufflation gases to the surgical cavity, the passage also configured to receive a medical instrument; and
a heating element disposed on or within at least a portion of the cannula along a longitudinal axis of the cannula,
wherein the heating element is configured to transfer heat to the insufflation gases passing through at least one of the cannula or a portion of the medical instrument to raise the temperature of the insufflation gases and/or the instrument so as to at least one of reduce or prevent condensation of one or both of the insufflation gases or on the medical instrument.

2. The surgical cannula of claim 1, wherein the heating element extends at least a partial length of the elongate shaft.

3. The surgical cannula of claim 2, wherein the heating element is disposed within a wall of the elongate shaft.

4. (canceled)

5. (canceled)

6. (canceled)

7. (canceled)

8. (canceled)

9. (canceled)

10. (canceled)

11. (canceled)

12. The surgical cannula of claim 1, wherein the heating element:
is isolated from the insufflation gases such that the heating element is out of an insufflation gases flow path;
extends at least substantially circumferentially around the hollow passage of the elongate shaft or the opening of the cannula upper housing;
extends helically along the elongate shaft or the cannula upper housing;
is flexible;
comprises an arcuate shape;
comprises a flexible band;
comprises a flexible PCB;
comprises a heater wire; or
comprises a thermo-elastic plastic material.

13. (canceled)

14. (canceled)

15. (canceled)

16. (canceled)

17. (canceled)

18. (canceled)

19. (canceled)

20. (canceled)

21. (canceled)

22. (canceled)

23. The surgical cannula of claim 1, comprising one or more electrical wires in electrical communication with the heating element, the one or more electrical wires extending one or both of along or through a wall of the cannula.

24. (canceled)

25. (canceled)

26. (canceled)

27. The surgical cannula of claim 1, wherein the heating element is powered by a controller of a humidifier, an independent controller, or a controller of an insufflator.

28. (canceled)

29. (canceled)

30. (canceled)

31. The surgical cannula of claim 1, wherein the elongate shaft comprises a plurality of lumens.

32. The surgical cannula of claim 31, wherein the heating element is disposed in one lumen or more lumens to heat gases passing through the one or more lumens.

33. The surgical cannula of claim 32, wherein the elongate shaft comprises two lumens and the heating element comprises first and second heating elements disposed in both lumens respectively.

34. The surgical cannula of claim 33, wherein the first heating element heats insufflation gases and the second heating element heats vented at least one of gases or smoke.

35. The surgical cannula of claim 1, comprising a filter disposed on or within the cannula.

36. The surgical cannula of claim 35, wherein the heating element is positioned so as to heat the filter.

37. (canceled)

38. A surgical cannula for providing insufflation gases to a surgical cavity and providing a passage for insertion of one or more medical instruments, the cannula comprising:
a cannula upper housing including an opening;
an elongate shaft extending from the cannula upper housing, the shaft defining a hollow passage to provide the insufflation gases to the surgical cavity, the passage also configured to receive a medical instrument; and
a heating element disposed on or within at least a portion of the cannula along a longitudinal axis of the cannula,
wherein the heating element is configured to increase a temperature of the insufflation gases passing through one or both of the cannula or the instrument above a dew point to one or both of reduce or prevent condensation of the gases or on the medical instrument.

39. The surgical cannula of claim 38, wherein the heating element extends at least a partial length of the elongate shaft.

40. The surgical cannula of claim 39, wherein the heating element is disposed within a wall of the elongate shaft.

41. (canceled)

42. (canceled)

43. (canceled)

44. (canceled)

45. (canceled)

46. (canceled)

47. (canceled)

48. (canceled)

49. The surgical cannula of claim 38, wherein the heating element is isolated from the insufflation gases such that the heating element is out of an insufflation gases flow path.

50. (canceled)

51. The surgical cannula of claim 38, wherein the heating element:
extends at least substantially circumferentially around the hollow passage of the elongate shaft or the opening of the cannula upper housing;
extends helically along the elongate shaft or the cannula upper housing; is flexible;
comprises a flexible band;
comprises an arcuate shape;
comprises a heater wire;
comprises a flexible PCB; or
comprises a thermo-elastic plastic material.

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. The surgical cannula of claim 38, wherein the heating element is powered by a controller of a humidifier, an independent controller, or a controller of an insufflator.

65. (canceled)

66. (canceled)

67. (canceled)

68. (canceled)

69. The surgical cannula of claim 38, wherein the heating element is disposed in one lumen or more lumens to heat gases passing through the one or more lumens.

70. The surgical cannula of claim 69, wherein the elongate shaft comprises two lumens and the heating element comprises first and second heating elements disposed in both lumens respectively.

71. The surgical cannula of claim 70, wherein the first heating element heats insufflation gases and the second heating element heats vented gases and/or smoke.

72. (canceled)

73. (canceled)

74. (canceled)

75. A surgical system for supplying insufflation gases to a surgical cavity, comprising:
a gases supply configured to provide the insufflation gases;
a humidifier in fluid communication with the gases supply and configured to humidify the insufflation gases received from the gases supply;
a surgical cannula according to any one of the preceding claims; and
a gases delivery tube extending between and in fluid communication with the humidifier and the surgical cannula, respectively,
wherein the gases delivery tube is in electrical communication with the humidifier and the surgical cannula, respectively, and
wherein the gases delivery tube directs the insufflation gases into the surgical cannula and directs an electrical current from the humidifier to the heating element within the surgical cannula.

76. (canceled)

77. (canceled)

78. (canceled)

79. (canceled)

80. (canceled)

81. (canceled)

82. (canceled)

83. (canceled)

84. (canceled)

85. (canceled)

86. The surgical system of claim 75, wherein the surgical cannula comprises a filter module that is removably coupled to or integrated with the surgical cannula.

87.-104. (canceled)
